(19) United States
(12) Patent Application Publication
(10) Pub. No.: US 2024/0299170 A1
(43) Pub. Date: Sep. 12, 2024

(54) INJECTABLE OR PERCUTANEOUS AUTOMATIC REPAIR DEVICE AND METHOD FOR INSERTING THE SAME

(71) Applicant: SHLOMO GABBAY, BOCA RATON, FL (US)

(72) Inventor: SHLOMO GABBAY, BOCA RATON, FL (US)

(21) Appl. No.: 18/277,469

(22) PCT Filed: Feb. 14, 2022
(86) PCT No.: PCT/US2022/016314

§ 371 Date: Aug. 16, 2023

Related U.S. Application Data
(60) Provisional application No. 63/150,826, filed on Feb. 18, 2021.

(51) Int. Cl. A61F 2/24

(52) U.S. Cl. CPC A61F 2/2463; A61F 2/2466; A61F 2230/0013; A61F 2230/0054

(57) ABSTRACT

An automatic repair device with oppositely oriented inflow and outflow portions may be positioned within an annulus of a heart valve. The outflow portion of the automatic repair device is shorter than the inflow portion and is asymmetrical, whereas the inflow portion is symmetrical. Both the inflow and outflow portions of the automatic repair device are shaped such that lateral pressure is not applied to the annulus after implantation is complete. The smaller outflow portion is especially shaped to conform to a mitral valve annulus and anterior leaflet to further reduce stress on adjacent cardiac structures after implantation. The automatic repair device is preferably constructed of memory shape material, such that the automatic repair device is compressible to fit within most catheters or trocars for implantation in the annulus. Upon implantation at the annulus, the automatic repair device automatically expands to return to its original shape and dimensions.

### BACKGROUND OF THE INVENTION

### Field of the Invention

The invention relates to cardiac prostheses, generally to fix, replace, or repair defective cardiac valves, and more specifically relates to an insertable automatic repair device to replace a defective posterior leaflet of a mitral valve to prevent significant regurgitation. The invention also relates to various methods of inserting such devices in a patient, including percutaneous insertion.

A heart valve may become defective or damaged, such as resulting from congenital malformation, disease, or aging. When the heart valve becomes defective or damaged, the leaflets may not function properly. One common problem associated with a degenerating heart valve is an enlargement of the valve annulus (e.g., dilation). This enlarges the opening between chambers of the heart, puts stress on heart valve leaflets, and creates small openings for blood to leak, or regurgitate, between chambers. Other problems that may result in valve dysfunction are chordal elongation and lesions developing on one or more of the leaflets.

The bicuspid valve, or mitral valve, is located in the left atrioventricular opening of the heart for passing blood unidirectionally from the left atrium to the left ventricle of the heart. The mitral valve is encircled by a dense fibrous annular ring and includes two valve leaflets of unequal size. A larger valve leaflet, known as the anterior leaflet, is located adjacent the aortic opening. The smaller leaflet is known as the posterior leaflet.

When a mitral valve functions properly, for example, the anterior and posterior leaflets coapt to prevent regurgitation of blood from the left ventricle into the left atrium when the left ventricle contracts. In order to withstand the substantial backpressure and prevent regurgitation of blood into the left atrium during the ventricular contraction, the anterior and posterior leaflets are held in place by fibrous cords, called cordae tendinae, that anchor the leaflets to the muscular wall of the heart.

By way of example, if an annulus enlarges or dilates to a point where the attached leaflets are unable to fully close, known as malcoaptation, regurgitation or valve prolapse may occur. Adverse clinical symptoms, such as chest pain, cardiac arrhythmias, dyspnea, may manifest in response to valve prolapse or regurgitation. As a result, surgical correction, either by valve repair procedures or by valve replacement, may be required.

Surgical reconstruction or repair procedures may include plication, chordal shortening, or chordal replacement to adjust the leaflets through reconstruction of the cordae tendinae. Another common repair procedure relates to remodeling of the valve annulus, e.g., annuloplasty, which may be accomplished by implantation of a prosthetic ring to help stabilize the annulus and to correct or help prevent valvular insufficiency which may result from defect or dysfunction of the valve annulus. Properly sizing and implanting the annuloplasty ring can substantially restore the valve annulus to its normal, undilated, circumference. In situations where the valve leaflets exhibit lesions, it also may be necessary to reconstruct one or more valve leaflets by securing grafts or patches to the leaflets, such as over lesions or holes formed in the leaflet.

All of these procedures involve reconstructing various structures of the heart valve, particularly the mitral valve, including the leaflets, fibrous cords, or the valve annulus. The repair or reconstruction of these structures is invasive, complicated, and time consuming, the results of which are not readily reproducible and vary from patient to patient.

### BRIEF DESCRIPTION OF THE RELATED ART

The intention of a cardiac prosthesis or replacement valve is usually to insert a new, functioning structure mimicking the original valve at the location of the old, defunct structure or valve, and to create a perfect seal at the implantation site such that ideally 100% of blood flow passes through the new valve and not through leaks along the circumference of the prosthesis, or along other areas.

U.S. Pat. No. 6,419,695 describes a cardiac prosthesis for improving operation of the heart. The prosthesis includes an annular base with a buttress extending generally axially from and inwardly relative to an arc portion of the annular base. The buttress provides a surface that a valve leaflet, usually the anterior leaflet of the mitral valve, may move into and out of engagement to control blood flow and reduce regurgitation.

U.S. Pat. No. 6,869,444 describes a cardiac prosthesis having an annular base with a buttress extending generally axially from and inwardly relative to an arc portion of the annular base. The cardiac prosthesis has a cross-sectional dimension that is variable between a reduced cross-sectional dimension and an expanded cross-sectional dimension to facilitate implantation.

U.S. Pat. No. 7,160,322 describes a cardiac prosthesis having an annular base with a buttress extending axially along a central axis of the annular base. The buttress is designed to provide a first surface for a first leaflet of a heart valve to move into and out of engagement with the buttress, and a second surface for a second leaflet of the heart valve to move into and out of engagement with the buttress. The design of this cardiac prosthesis addresses prolapse of the heart valves, such as the mitral leaflets prolapsing from the left ventricle into the left atrium.

Artificial valve implants, including either mechanical valve or synthetic/biological valves, have long used lateral pressure to remain along a fixed position within a patient after implantation. Most such valves are still implanted surgically (opening the patient's sternum, open chest cavity, pulmonary bypass, etc.). Mechanical valves cannot be implanted percutaneously, as they cannot be compressed, and therefore must be implanted through an open chest cavity.

Transcatheter Aortic Valve Implantation (TAVI) devices include synthetic or biological tissue serving as replacement leaflets that are held within a frame. These devices are implanted percutaneously, which does not require an open cavity, just a catheter through an opening in the patient's skin, to substitute for calcified aortic valves and in a small number of pulmonary valves. Before implantation, TAVI devices are laterally compressed or reduced along their diameter, to fit within a catheter or similar device for implantation. Once the TAVI device has reached an implantation site within a patient, the implant is deposited within the artery or similar structure, typically with calcification to provide a more solid, stable surface to secure the TAVI device. Generally, the heart structure in which the TAVI device is situated has a smaller diameter than the fully expanded implant. The lateral pressure created through the TAVI device not being able to fully expand provides a counterbalancing force, with the intention that the counterbalancing force keeps the TAVI device situated in the implantation site.

In early cardiac valve replacement devices, such as provided in US 2003/0040792, such devices where implanted within the old valve. For instance, if the device was designed to replace an aortic valve, the support would be inserted within the old, original valve, such that the old aortic valve formed essentially a concentric ring around the support. The new valve, synthetic or biological, would then be positioned ideally at the same position within the aorta as the original valve. However, the old, original valve tissue was not removed, and additional pressure would be created within the cardiac system. This old valve tissue would be compressed between the support of the device and the wall of the aorta. This causes the diameter of the aorta to expand a bit around the area of the implantation site and the old valve. In the aorta, the negative effects of such expansion is minimized. Further, a tolerable amount of expansion around certain cardiac valves, such as the aortic valve, is not immediately dangerous to the patient due to the positioning of those valves relative to other cardiovascular structures.

Replacing, or inserting, prostheses in other cardiovascular valves require diligent attention to any expansion or additional pressure, as outward radial pressure in such valves causes further pressure to be applied to adjacent structures. This is true of the mitral valve. The mitral valve has a relatively larger diameter than the aortic valve. Most mitral valve replacement devices produced by major industry leaders in valve surgery devices have used the same technology that is used previously in aortic valves. This means that current mitral valve devices rely on calcified tissue to provide a local site for devices employing lateral pressure to fix the device in place. The mitral valve annulus is generally not calcified, nor does it usually become sufficiently calcified, and is instead typically soft due to important anatomical differences. The aortic valve has a smaller diameter than the mitral valve, is circular, becomes calcified, and the arterial nature of the aorta's tube is stronger. The mitral valve annulus is 60% soft muscle and only anteriorly fibrotic and non-distendable. Worse yet, the left circumflex coronary artery is positioned 1 to 2 millimeters (mm) from the border of the posterior leaflet. Any mitral valve design that is based on lateral pressure risks occluding the coronary artery, which will kill the patient. However, if pressure is too low, the blood will flow around the valve or will totally dislodge the valve, again causing death.

U.S. Pat. No. 8,845,722 describes cardiac prosthesis having a valve portion supported within a support structure. The support structure has an inflow portion and an outflow portion extended radially outwardly from a central annular portion to create to oppositely oriented umbrellas of equal size and proportion. The valve portion is secured with the central annular portion. This cardiac prosthesis helps to address the inherent issues in prostheses relying on lateral pressure. The structure of this cardiac prosthesis clamps the old valve tissue between the in-flow and out-flow members of the device around the circumference of the old valve and device. The device, including the new valve, is therefore secured around the old valve in such a way that blood flow through the new valve is optimized and, more importantly, the device does not impart any lateral force or pressure on the surrounding cardiac tissue.

In most patients, only one of the two leaflets of the mitral valve is deficient. In 85 percent of patients with mitral valve deficiencies, the posterior leaflet is deficient and the anterior leaflet remains structurally sufficient. Moreover, the anterior leaflet accounts for 70 percent of the surface area of the mitral valve when closed. However, the uniform umbrella of the outflow portion of the prosthesis of U.S. Pat. No. 8,845,722 does not fully account for the structure of the mitral valve leaflets. The umbrella of the inflow portion attaches to atrial tissue in the left atrium. However, the umbrella of the outflow portion, if equal in size and proportion, extending into the left ventricle from the left atrioventricular opening covers a portion of both the posterior leaflet and the anterior leaflet. This restricts movement of the otherwise healthy valve.

There is therefore a need in the art to provide an insertable repair structure that does not occlude an operational valve leaflet, such as the anterior leaflet of the mitral valve, and does not apply excessive lateral pressure around a valve annulus.

### SUMMARY OF THE INVENTION

According to a preferred embodiment of the invention, an automatic repair device comprises a fixation support member having an annular support frame formed from a plurality of support features, the plurality of support features including inflow support features and outflow support features, the inflow support features extending in an inflow direction from a circumferential path and the outflow support features extending in an outflow direction from the circumferential path, and a support member web at least partially encasing the annular support frame to provide at least an inflow contact surface and an outflow contact surface, wherein the outflow support features are shorter than the inflow support features; and a buttress having a buttress support frame, and a buttress web at least partially encasing the buttress to form a surface against which at least one leaflet of a heart valve may engage the buttress, wherein the buttress is secured to the annular support frame along the circumferential path such that the buttress extends in the outflow direction; wherein the fixation support member and buttress are flexible between a compressed configuration and an expanded configuration.

According to another embodiment of the invention, an automatic repair device comprises a buttress apparatus having a generally arcuate base portion and a buttress, the buttress apparatus having an inflow end and an outflow end, the buttress extending generally radially inwardly and axially along the outflow end relative to the base portion so as to permit substantially bi-directional flow of blood axially relative to the buttress apparatus, the buttress having a surface dimensioned and configured to be engaged by at least one leaflet of a heart valve when the automatic repair device is implanted at the heart valve, whereby when the automatic repair device is implanted at the heart valve, movement of the at least one leaflet of the heart valve relative to the surface of the buttress provides substantially unidirectional flow of blood relative to the apparatus; and a fixation support member secured along a circumference of the arcuate base portion of the buttress apparatus, the fixation support member comprising inflow and outflow portions, the inflow portion of the fixation support member extending from a radially inner contact surface of the fixation support member radially outwardly and axially in a direction of the inflow end of the buttress apparatus upon deployment of the automatic repair device, the outflow portion of the fixation support member extending from the radially inner contact surface radially outwardly and axially in a direction away from the inflow portion of the fixation support member upon deployment of the automatic repair device, the buttress apparatus and the fixation support member being deformable between a reduced cross-sectional dimension and an expanded cross-sectional dimension thereof when the automatic repair device is deployed, whereby implantation of the automatic repair device is facilitated, wherein the fixation support member comprises an annular frame support formed from a plurality of support features, the support features alternate extending between the inflow and outflow directions along a circumferential path corresponding to the radially inner contact surface, the support features being interconnected at first junctures at an axial extent of the inflow portion and being interconnected at second junctures at an axial extent of the outflow portion, each support feature being directly connected to only two other support features such that the support features collectively extend along the entire axial length of the fixation support member, and wherein the support features extending in the outflow direction are shorter than the support features extending in the inflow direction, such that a diameter of the outflow portion of the fixation support member is smaller than the diameter of inflow portion of the fixation support member.

According to another embodiment of the invention, an automatic repair device, comprises a fixation support member having an inflow portion and an outflow portion, the inflow portion having an outer inflow circumference and an inner inflow circumference with an inflow contact surface defined there between and extending radially outwardly and axially from the inner inflow circumference to the outer inflow circumference when the automatic repair device is in a deployed configuration, the outflow portion having an outer outflow circumference and an inner outflow circumference with an outflow contact surface defined there between and extending radially outwardly and axially from the inner outflow circumference to the outer outflow circumference when the automatic repair device is in a deployed configuration, wherein the inflow portion and the outflow portion are coaxially secured along the inner inflow circumference and the inner outflow circumference to form a circumferential path between the inflow portion and outflow portion, such that the fixation support member has a generally hyperboloidal shape in the deployed configuration, and a buttress extending generally radially inwardly and axially along the outflow portion relative to the circumferential path, the buttress having a surface dimensioned and configured to be engaged by at least one leaflet of a heart valve when the automatic repair device is implanted at the heart valve, whereby when the automatic repair device is implanted at the heart valve, movement of the at least one leaflet of the heart valve relative to the surface of the buttress provides substantially unidirectional flow of blood relative to the apparatus, wherein the fixation support member and buttress are deformable from the deployed configuration to any other configuration and automatically expandable again to the deployed configuration.

According to an embodiment of a method for implanting an automatic repair device, the method comprises compressing the automatic repair device to reduce an overall diameter of the automatic repair device; loading the compressed automatic repair device into an implanter; inserting the loaded implanter into a heart of a patient; depositing the compressed automatic repair device into an annulus of a heart valve; expanding the automatic repair device in the annulus such that the automatic repair device is implanted in the annulus; and removing the implanter from the patient.

Another embodiment of the method further includes the automatic repair device comprising a fixation support member having an annular support frame formed from a plurality of support features, the plurality of support features including inflow support features and outflow support features, the inflow support features extending in an inflow direction from a circumferential path and the outflow support features extending in an outflow direction from the circumferential path, and a support member web at least partially encasing the annular support frame to provide at least an inflow contact surface and an outflow contact surface, wherein the outflow support features are shorter than the inflow support features; a buttress having a buttress support frame, and a buttress web at least partially encasing the buttress to form a surface against which at least one leaflet of a heart valve may engage the buttress, wherein the buttress is secured to the annular support frame along the circumferential path such that the buttress extends in the outflow direction; wherein the fixation support member and buttress are flexible between a compressed configuration and an expanded configuration.

Another embodiment of the method teaches the implanter is a catheter or trocar.

A further embodiment of the method teaches the loaded implanter is inserted through an apex of the heart.

Another embodiment of the method teaches the automatic repair device automatically expands in the annulus after deposited in the annulus.

According to another embodiment of the invention, an automatic repair device comprises a fixation support member having an annular support frame formed from a plurality of support features, the plurality of support features including inflow support features defining an inflow portion and outflow support features defining an outflow portion, the inflow support features extending in an inflow direction from a circumferential path and the outflow support features extending in an outflow direction from the circumferential path, and a support member web at least partially encasing the annular support frame to provide at least an inflow contact surface and an outflow contact surface, wherein the outflow support features are shorter than the inflow support features, the outflow support features vary in length, and the intflow support features are uniform in length; a buttress having a buttress support frame, and a buttress web at least partially encasing the buttress to form a surface against which at least one leaflet of a heart valve may engage the buttress, wherein the buttress is secured to the annular support frame along the circumferential path such that the buttress extends in the outflow direction; wherein the fixation support member and buttress are flexible between a compressed configuration and an expanded configuration.

Another embodiment of the automatic repair device includes, along the circumferential path, the outflow support features shorten in length and then lengthen such that a shortest outflow support feature is oppositely oriented from a longest support feature.

According to another embodiment of the invention, an automatic repair device comprises a fixation support member having an inflow portion and an outflow portion each extending radially outwardly and oppositely from a central annular portion to create an inflow umbrella and an outflow umbrella, wherein the inflow umbrella extends uniformly from the central annular portion, and wherein the outflow umbrella does not extend uniformly from the central annular portion and an overall length of the outflow umbrella is shorter than the inflow umbrella; and a buttress, having a contact surface, secured along the central annular portion, the buttress extending away from the central annular portion and within the outflow umbrella, wherein the fixation support member and buttress are flexible between a compressed configuration and an expanded configuration.

### BRIEF DESCRIPTION OF THE DRAWINGS

A better understanding of the invention will be had with respect to the accompanying drawings wherein:

FIG. 1 is a perspective view of a support frame embodiment for an automatic repair device, the frame being a single, continuous structure;

FIG. 2A is a side view along the support frame shown in FIG. 1;

FIG. 2B is a side view along an outflow portion of the support frame shown in FIG. 1;

FIG. 2C is a side view along an outflow portion of an alternative support frame embodiment;

FIG. 3A is a top view of the support frame shown in FIG. 1;

FIG. 3B is a top view of an alternative support frame embodiment, such as shown in FIG. 2C;

FIG. 4A is a cross-sectional view of an inflow support arm and corresponding spike of the support frame of FIG. 1;

FIG. 4B is a side view of an outflow support arm and corresponding spike of the frame of FIG. 1;

FIG. 5 is a back perspective view of a buttress frame, the buttress frame being a single, continuous structure;

FIG. 6 is a side perspective view of the buttress frame of FIG. 5;

FIG. 7 is a top view of the buttress frame of FIG. 5;

FIG. 8 is a back view of the buttress frame of FIG. 5;

FIG. 9 is a back view of a buttress formed from a covering around the buttress frame of FIG. 5;

FIG. 10 is a bottom perspective view of a buttress embodiment having the buttress of FIG. 9 and a buttress curtain forming an opening;

FIG. 11 is back perspective view of the buttress embodiment of FIG. 10;

FIG. 12 is a side view of an automatic repair device embodiment having the support frame of FIG. 1;

FIG. 13 is a side view of an automatic repair device embodiment having the support frame of FIG. 1 and the buttress of FIG. 10;

FIG. 14A is a cross-sectional view of the automatic repair device of FIG. 13;

FIG. 14B is a cross-sectional view of an automatic repair device embodiment having a buttress of FIG. 9 without a buttress curtain;

FIG. 15 is a perspective view of an alternative support frame embodiment for an automatic repair device, the support frame being two separate structures securable together, each said structure being continuous;

FIG. 16 is an illustration of percutaneous insertion of an automatic repair device according to an embodiment of the invention via an apex of a heart;

FIG. 17 is an illustration of an automatic repair device insertion according to another embodiment of the invention via an atrium of the heart;

FIG. 18 is an illustration of the automatic repair device insertion of FIG. 16, showing the operator using a figure to guide the automatic repair device into a proper positioning during expansion of the automatic repair device in a heart valve annulus;

FIG. 19 is an illustration of the automatic repair device insertion of FIG. 17, showing expansion of the automatic repair device in a heart valve annulus;

FIG. 20A is an illustration of an implantation device containing an automatic repair device embodiment, such as shown in FIG. 14A, in a cross-sectional view of an end enclosure;

FIG. 20B is a cross-sectional view of an alternative end enclosure of an implantation device as shown in FIG. 20A;

FIG. 21 is an illustration of an implantation device embodiment of FIG. 20A being implanted percutaneously through the apex of the heart;

FIG. 22A is an illustration of the implantation device embodiment of FIG. 21 being implanted percutaneously through a fossa ovalis dividing right and left atrium;

FIG. 22B is an illustration of the implantation of FIG. 22A showing the implantation device extending up through the inferior vena cava, into the right atrium, through an opening corresponding to the fossa ovalis, through the left atrium, and into the mitral valve annulus;

FIG. 23 is an illustration of blood flow in a representative heart through the automatic repair device embodiment from the left atrium into the left ventricle during diastole;

FIG. 24 is an illustration blood flow from the left ventricle into the aorta during systole, with the anterior leaflet of the mitral valve coapting with the buttress of the automatic repair device to blocking blood flow from the left ventricle back into the left atrium;

FIG. 25 is a cross-sectional view of an automatic repair device embodiment, with the embodiment shown in FIG. 14A shown as an example, implanted around the mitral valve annulus as blood flows from the left atrium to the left ventricle; and

FIG. 26 is a cross-sectional view of the automatic repair device embodiment of FIG. 25 implanted around the mitral valve annulus, where an anterior leaflet coapts against a buttress to prevent regurgitation as blood flows from the left ventricle to the aorta.

A further understanding of the invention and its embodiments will be had in reference to the detailed description.

### DETAILED DESCRIPTION OF THE INVENTION

It will be appreciated that numerous specific details have been provided for a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description is not to be considered so that it may limit the scope of the embodiments described herein in any way, but rather as merely describing the implementation of the various embodiments described herein.

The description that follows, and the embodiments described therein, are provided by way of illustration of an example, or examples, of particular embodiments of the principles of the present invention. These examples are provided for the purposes of explanation, and not limitation, of those principles and of the invention. It will also be appreciated that similar structures between embodiments are marked with identical reference numbers for ease of reference.

The invention relates generally to an automatic repair device that includes a fixation support member dimensioned and configured to facilitate implantation of the automatic repair device within and around a heart valve annulus using low invasive procedures. The automatic repair device is purposed for implantation at an atrio-ventricular position, namely, at the bicuspid valve position. The embodiments of the automatic repair device and implantation methods described herein, however, can also be utilized for replacement of other heart valves and structures beyond the bicuspid valve.

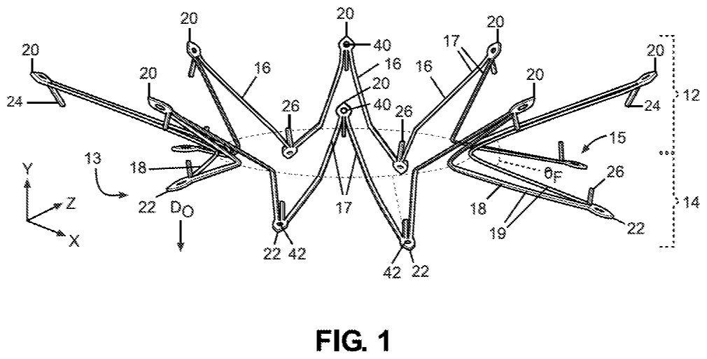

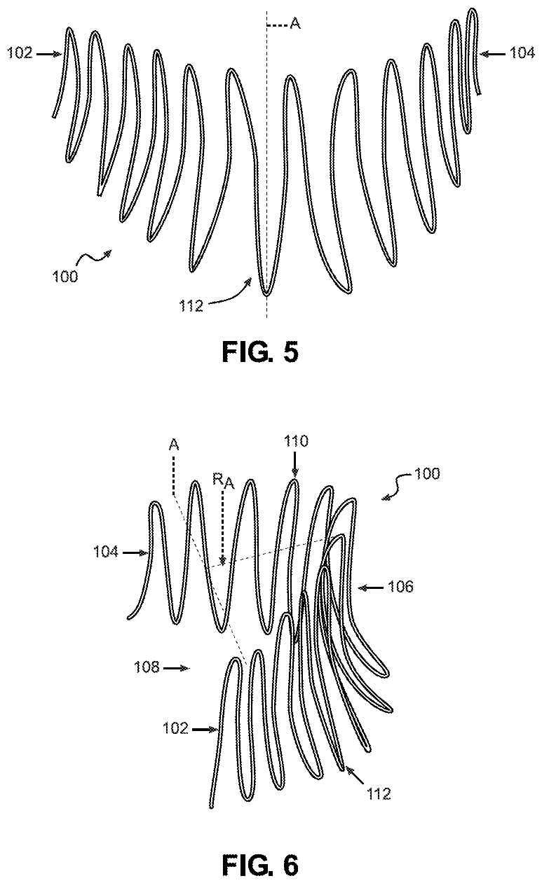

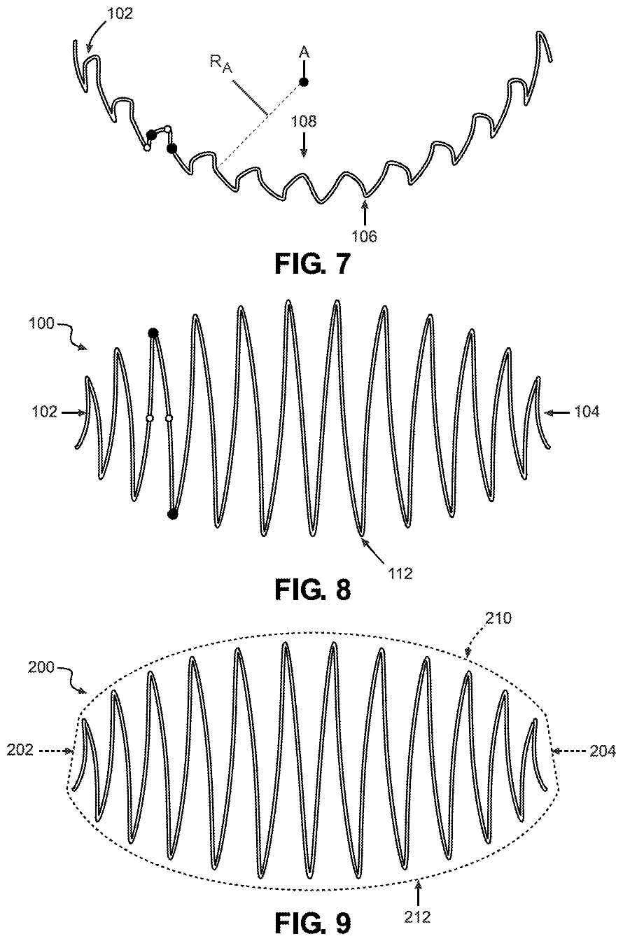

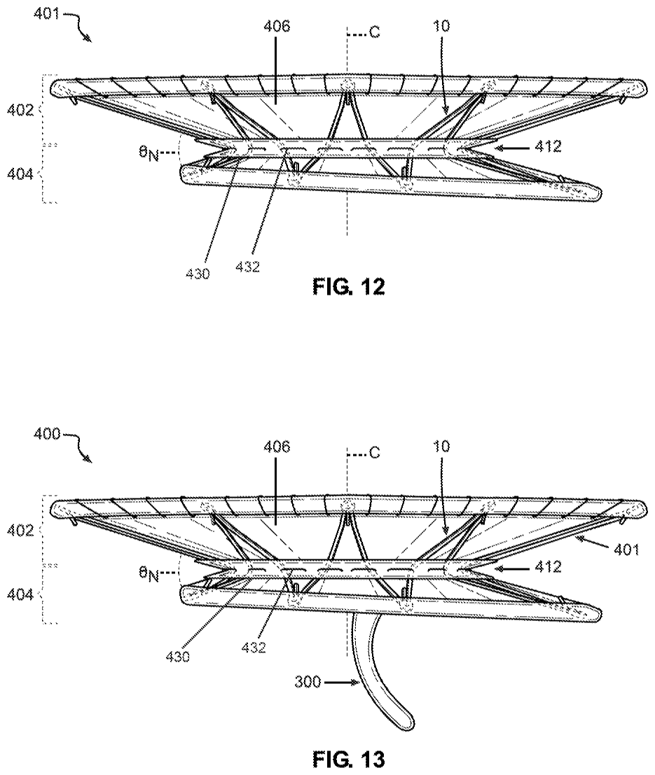

FIG. 1 depicts an embodiment of an annular support frame 10 that can be utilized in an automatic repair device 400 embodiment for correcting or repairing deficiencies in heart valves. In this embodiment, the annular support frame 10 is utilized to anchor or help hold the automatic repair device in a desired axial position relative to a heart valve annulus when implanted. The support frame 10 is utilized to form a fixation support member 401, such as shown in FIG. 12. A buttress support frame 100, shown in FIGS. 5-8, supports and forms a buttress, such as buttress embodiments 200 or 300, shown in FIGS. 9-11. Together, the fixation support member 401 and buttress 200 or 300 form the automatic repair device 400. As used herein, the support frame 10 may be alternatively referred to as a support structure and the buttress support frame 100 may be referred to as a buttress support.

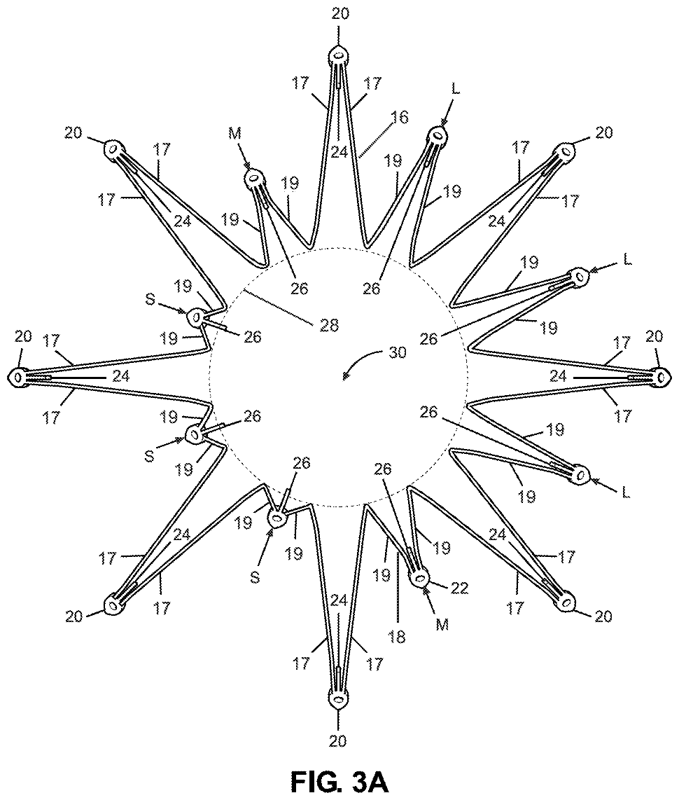

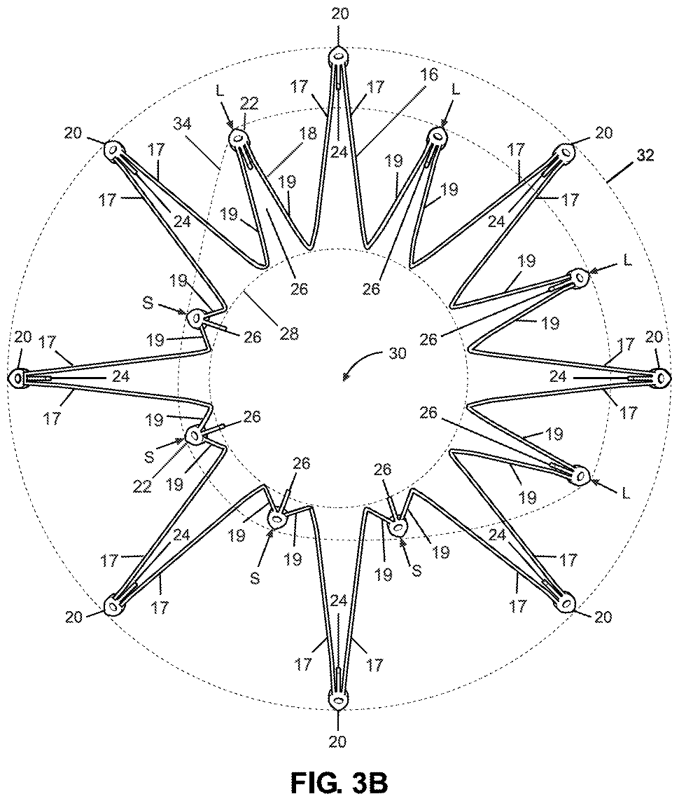

Regarding the support frame 10 embodiment shown in FIGS. 1-4B, the support frame is implemented as a flexible and deformable annular support structure that can be deformed to a reduced cross-sectional dimension and then automatically expand to its original, fully expanded, cross-sectional dimension and shape, such as shown in FIG. 1. The support frame 10 includes an inflow portion 12 and an outflow portion 14. An annular opening 30, better shown in FIG. 3A, extends through the support frame 10 corresponding to a radially inner extent thereof, indicated at dashed line 28, of the support frame.

The inflow portion 12 includes a plurality of support features 16 and outflow portion 14 includes a plurality of support features 18. The inflow support features 16 extend radially outwardly and axially from the inner extent 28. The inflow support features 16 extend away from an inflow direction DI, which indicates the intended direction of blood flow along the inflow portion 12 of support frame 10 after implantation. The outflow support features 18 extend radially outwardly and axially from the inner extent 28. The outflow support features 18 extend toward an outflow direction D_{O}, which is the direction of intended blood flow along the outflow portion 14 after implantation. Therefore, the inflow support features 16 extend in an opposite axial direction from the outflow support features 18, as overall intended blood flow through the support frame 10 and automatic repair device 400 is unidirectional (i.e. the inflow direction D_{I} is the same as the outflow direction D_{O}).

Each of the inflow support features 16 extends from the radially inner extent 28 and terminates at corresponding distal end 20 thereof. Each of the outflow support features 18 also extends from the radially inner extent 28 to terminate in a respective distal end 22. While both the inflow support features 16 and the outflow support features 18 extend from the radially inner extent 28, they extend in opposite directions from the radially inner extent.

Each inflow support feature 16 of the plurality of inflow support features includes a pair of inflow support arms 17. The pair of inflow support arms extends from the radially inner extent 28 and toward each other until they are interconnected at a juncture at the corresponding distal end 20. The respective juncture, or distal end 20, can be biased (e.g., configured as springs) to urge each of the associated inflow support arms 17 apart to maintain the support frame 10 in its expanded condition. The pair of inflow support arms may 17 be individual structures secured together as known in the art, or the pair of inflow support arms and distal end 20 of each inflow support feature 16 may be formed from a continuous structure, such as wire or metal bent, formed, or cut to create such a structure.

Each outflow support feature 18 of the plurality of outflow support features include a pair of outflow support arms 19. The pair of outflow support arms 19 extend from the radially inner extent 28 and toward each other until they are interconnected at a juncture at the corresponding distal end 22. The respective juncture, or distal end 22, can be biased (e.g., configured as springs) to urge each of the associated outflow support arms 19 apart to maintain the support frame 10 in its expanded condition. The pair of outflow support arms 19 may be individual structures secured together as known in the art, or the pair of outflow support arms and distal end 22 of each outflow support feature 18 may be formed from a continuous structure, such as wire or metal bent, formed, or cut to create such a structure.

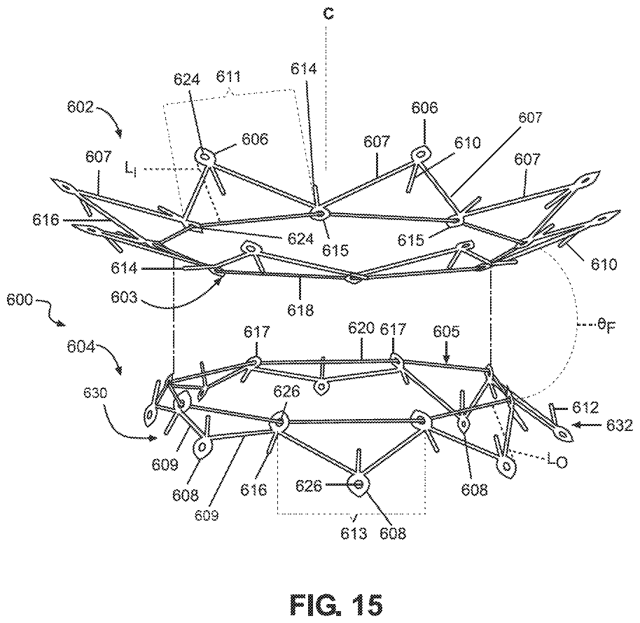

In the support frame 10 embodiment provided in FIG. 1, there are eight inflow support features 16 and eight outflow support features 18. Those skilled in the art will understand and appreciate that other amounts (e.g., 6, 7, 9, 10, etc.) of inflow support features 16 and outflow support features 18 may be included in the support frame 10. However, in an embodiment where the inflow support features 16 and outflow support features 18 are continuous with each other, discussed in further detail herein, there must be an equal amount of inflow support features and outflow support features. In other embodiments of a support frame 10 that is formed of two or more separate structures, i.e. FIG. 15, the number of inflow support features 16 and outflow support features 18 need not be identical.

Each distal end 20 preferably has an eye or a hole 40 for receiving and securing string, line, or similar suture material. Each distal end 22 preferably has an eye or a hole 42 likewise for receiving and securing string, line, or similar suture material.

A preferred embodiment of the support frame 10 is configured as a continuous monolithic structure, as shown in the embodiments of FIGS. 1-3B. Along and from the inner radial extent 28, a single piece of material is shaped, or multiple pieces are secured together, to form alternating connected inflow support features 16 and outflow support features 18. By way of example, the support frame 10 can be formed from a single loop or circular wire material, such as from materials commonly used to make stents, which is then bent and formed according to the support frame of FIGS. 1-3B and without being cut or otherwise separated. Alternatively, the two or more pieces of material may be attached together as known in the art to form the continuous monolithic structure. The support frame 10 is preferably made from a material with shape memory properties, such as Nitinol, that allows the support frame to be deformed from the natural or original shape and then automatically return to the natural or original shape.

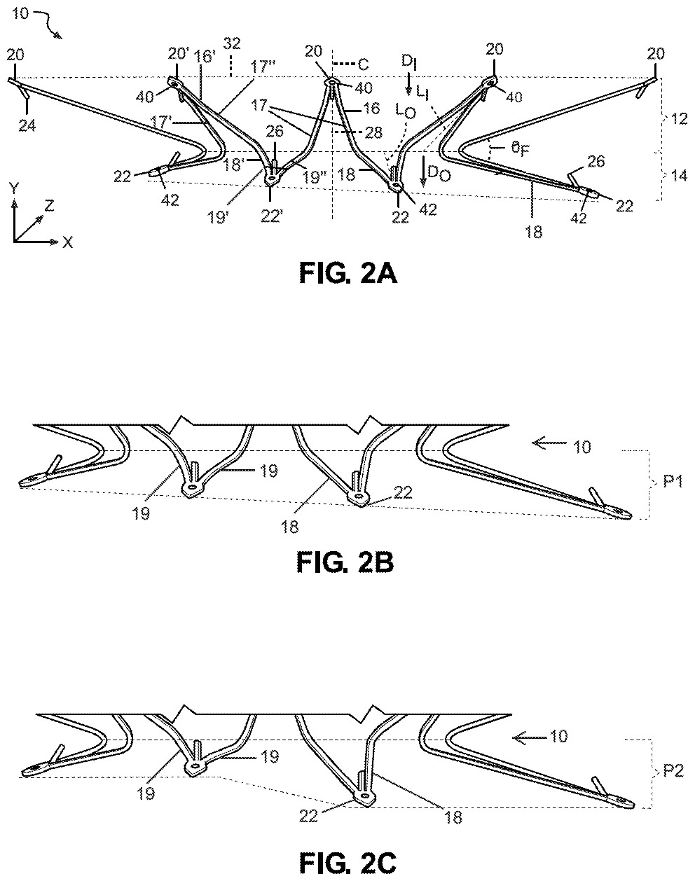

The support frame 10 is described herein as having a zig-zag arrangement. The zig-zag arrangement of the support frame 10 means that the structure of the support frame 10 alternates extending along the inflow portion 12 and the outflow portion 14. Specifically, in regards to the embodiment of FIGS. 1-4B, the structure of the support frame 10 alternates between an inflow distal end 20 to an outflow distal end 22 to an inflow distal end to an outflow distal end, etc. This can alternatively be described as alternating between a pair of inflow support arms 17 forming an inflow distal end 20 and pairs of outflow support arms 19 forming an outflow distal end 22, or as alternating between an inflow support feature 16 and an outflow support feature 18. For example, starting along the support frame 10 at the radially inner extent 28, oriented about axis C as shown in FIG. 2, a first inflow support arm 17′ extends radially outwardly along a z-axis, axially along a y-axis, and horizontally along an x-axis from the inner extent 28 and opposite to the inflow direction DI. This inflow support arm 17′ forms an initial inflow distal end 20 where it meets an adjacent inflow support member 17″ also extending radially outwardly along the z-axis, axially along the y-axis, and horizontally along the x-axis from the inner extent 28 and opposite to the inflow direction DI. However, the initial inflow support arm 17′ and the adjacent inflow support arm 17″, which together form an initial pair of inflow support arms or initial inflow support feature 16′, extend along the x-axis in opposite directions and toward each other such that they meet at and form the initial distal end 20′. At the radially inner extent 28, the adjacent inflow support arm 17″ is connected to and continuous with an initial outflow support arm 19′ extending radially outwardly along the z-axis in the same z-axis direction as the initial pair of inflow support features, axially along the y-axis in an opposite direction to the initial pair of inflow support features, and horizontally along the x-axis in the same direction is the initial inflow support arm 17′, but opposite to the x-axis direction as the adjacent inflow support arm 17″. The initial outflow support arm 19′ forms an initial outflow distal end 22′ where it meets an adjacent outflow support arm 19″ extending in the same z- and y-axis direction, but opposite x-axis direction, forming an initial pair of outflow support features or initial outflow support feature 18′. This pattern is then repeated with further sets of an inflow support features 16 and outflow support features 18 about the radially inner extent 28 until returning to the initial inflow support arm 17′.

The zig-zag arrangement of the support frame 10 allows flexibility, deformation, and reformation of the support frame. This flexibility allows the support frame 10 to form and maintain the annular opening 30 at a natural or original diameter preferred and sized for proper operation and blood flow once the automatic repair device 400 is implanted in a patient. Alternatively, the annular opening 30 can be reduced from the natural diameter to a desired smaller diameter by reducing the radially inner extent 28, such as by a suture or other means. This shape also allows the completed automatic repair device to rely on a grip mechanism, or clamping between the inflow and outflow portions 12 and 14, instead of on lateral pressure used relied upon in other devices currently available.

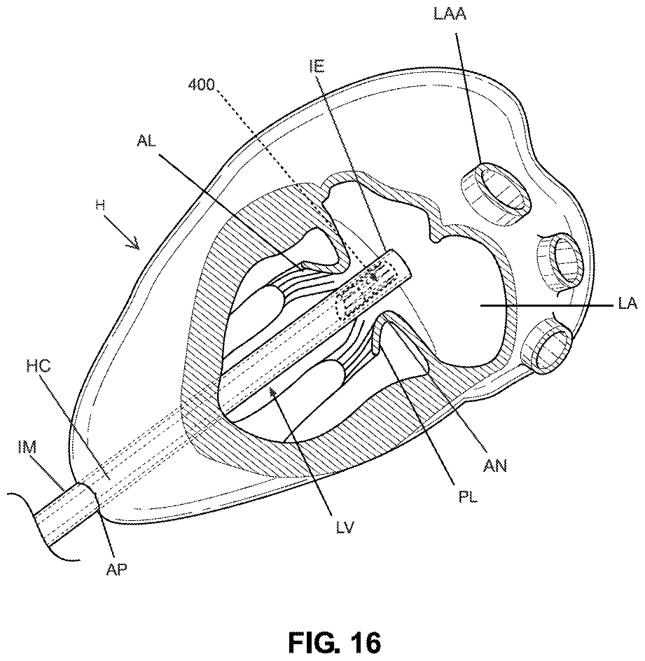

Further, the support frame 10 can be manually positioned in a horizontally deformed state such that a diameter of an inflow circumference 32 formed along the plurality of inflow distal ends 20, a diameter of an outflow circumference 34 formed along the plurality of distal ends 22, and the diameter of the annular opening 30 are all equal, wherein the support frame forms a cylindrical or tubular shape. As the overall diameter of the support frame 10 can be greatly reduced in this deformed shaped, this is especially beneficial for loading the automatic repair device 400 into a catheter, trocar, or similar implantation device for insertion into the heart and delivery of the automatic repair device to the implantation site, such as provided in FIGS. 16-19 and 21-22B. FIGS. 3A-3B depict embodiments of the support frame 10 along a top view and provide a relational views and corresponding position of the inflow circumference 32, outflow circumference 34, and inner radial extent 28.

The support frame 10 can also be manually positioned in a vertically deformed state, wherein the one or more of the inflow support arms 17 and/or outflow support arms 19 outwardly extend toward a plane defined by the inner radial extent 28. In this deformed position, the support frame 10 is substantially flattened along the x-axis. In this manner, the inflow circumference 32 and outflow circumference 34 may be changed while maintaining or changing the circumference of the radially inner extent 28.

Due to the structural arrangement of support arms 17 and 19 that define the inflow support features 16 and outflow support features 18, and ultimately the inflow and outflow portions 12 and 14, it is thus shown that the support frame 10 provides a generally hourglass shape in its natural, operational, or original position in which it has a substantially V-shaped cross-sectional configuration along a longitudinal cross-section taken radially outwardly through one side of the fixation support in its resting configuration. This substantially V-shaped cross-sectional configuration can include a V-shaped configuration, a U-shaped configuration, or a configuration between two such shapes.

An angle Θ_{F} measured between the inflow portion 12 and outflow portion 14 is preferably between 20-45 degrees (°), inclusive, in the operational configuration of the support frame 10 to properly conform to the shape of the mitral valve annulus. However, it is conceivable that the angle Θ_{F} could be outside of this range to accommodate specific patient conditions or shapes of other valve annuli. The angle Θ_{F} is consistent about the support frame 10 for a given embodiment, but may be different between embodiments or due to a specific shape and size of the valve annulus of a given patient.

An important feature of the support frame 10 is the difference in lengths between the inflow support feature 16 and outflow support feature 18. Namely, an inflow support feature 16 is longer than an outflow support feature 18. The inflow portion length L_{I} is measurable along any inflow support feature 16, and is defined between the distal end 20 of the inflow support feature and a midpoint between each inflow support arm 17 of the inflow support feature along the radial inner extent 28. The inflow portion length L_{I} is longer than any given outflow portion length L_{O}, which is defined between the distal end 22 of an outflow support feature 18 and a midpoint between each outflow support arm 19 of the outflow support feature along the radial inner extent 28. Each inflow support feature 16 of a given support frame 10 shares the same length L_{I}, although the value of L_{I} may vary between different embodiments of the support frame. This creates an inflow portion 12 that is uniform in shape and size along its circumference.

Another important feature of the support frame 10 is that the length L_{O} varies between two or more groups of outflow support features 18 of the outflow portion 14. Shorter outflow support features 18 are located together along one side of the outflow portion 14, instead of being interspersed among longer outflow support features. The outflow support features 18 may gradually change in length L_{O} along adjacent outflow support features as shown by dashed line P1 in FIG. 2B. Alternatively, there may be two groups of outflow support features 18, each group having its own set value for length L_{O}, to create a group of short outflow support features and a group of long outflow support features, shown along a side profile as an irregular hexagon illustrated by a dashed line P2 in FIG. 2C.

Two different embodiments of the support frame 10 are shown between FIGS. 3A and 3B to demonstrate possible orientations of outflow support features 18 with different lengths. In the support frame 10 of FIG. 3A, there are three different lengths L_{O} of outflow support features 18, with group S outflow support features being the shortest or having a lowest value of L_{O}, group L outflow support features being the longest or having a largest value of L_{O}, and group M outflow support features having a value between the L_{O} of group S and L. The support frame 10 of FIG. 3B shows a group S of outflow support features 18 with one value of L_{O} and a group L of outflow support features having another value of L_{O}, with the group L value of L_{O} being larger than the group S value of L_{O}.

The support frame 10, and automatic repair device 400 by extension, does not apply unwanted lateral pressure to certain cardiac structures when inserted within a valve annulus, such as the mitral valve annulus. As discussed, outward lateral pressure around the mitral valve, especially on the ventricular side of the mitral valve can lead to death by occlusion of the coronary artery. The support frame 10 allows the heart valve to be secured in the mitral valve using axial pressure, which involves the annulus being clamped between the inflow portion 12 and outflow portion 14 via the inflow support features 16 and outflow support features 18, respectively. Such vertical forces do not present the same health risks as the outward horizontal forces previously discussed. Further, the uneven outflow portion 14 allows the support frame 10 to be positioned such that a shorter area 13 of the outflow portion is oriented toward a properly-functioning valve leaflet. The properly-functioning leaflet can freely move toward axis C of the support frame 10 via the shorter area 13 to partially close the annular opening 30. A longer area 15 of the outflow portion can be positioned over a non-functioning leaflet of the valve to be repaired.

As previously discussed, the outflow portion 14 is overall shorter than the inflow portion 12. Therefore, preferably, the longest outflow support arm 19 length L_{O} is therefore two-thirds of the inflow support arm 17 length L_{I}. However, embodiments of the support frame 10 and automatic repair device 400 envision other embodiments wherein L_{O}<L_{I}.

To aid in the attachment of the automatic repair device 400 to a valve annulus, each inflow support feature 16 and outflow support feature 18 preferably includes one or more projections or spikes 24 and 26, respectively, located along distal ends 20 and 22. The inflow spikes 24 are shaped and oriented differently from the outflow spikes 26 to further improve and secure implantation of the automatic repair device 400. An inflow spike 24 extends from each inflow distal end 20 axially toward the outflow portion 14 and away from the corresponding inflow support feature 16, while an outflow spike 26 extends from each outflow distal end 22 axially toward the opposing inflow portion 12 and away from the corresponding outflow support feature 18. Various numbers and arrangements of spikes 24 and 26 can be implemented, such as single spike or more than two spikes at some or all of the distal ends 20 and 22. Additionally, ends of the spikes 24 and 26 can have tapered or sharpened and/or barbed tips to facilitate gripping surrounding tissue when implanted.

The inflow spikes 24 are preferably shorter and flatter than the outflow spikes 26. Preferably, each inflow spike 24 has an angle Θ₁ of 30° measured between the inflow spike and the inflow support feature 16 to which it is attached. Further, each inflow spike 24 has a length L_{S1}. Each outflow spike 26 preferably has an angle Θ₂ of 45° measured between the outflow spike and the outflow support feature 18 to which it is attached. Each outflow spike 26 has a length L_{S2}. Preferably, the outflow spike 26 is 2 mm longer than the inflow spike 24, or L_{S2}=L_{S1}+2 mm. The specific values of L_{S1} and L_{S2} may vary as appropriate to safely secure the support frame and automatic repair device within a patient.

FIGS. 5-8 provide different views of a buttress frame 100 embodiment. When covered, the buttress frame 100 provides shape to a buttress 200 or 300 embodiment, which forms the automatic repair device 400, along with a covered support frame 10 embodiment, or automatic repair device body 401. In a preferred embodiment, the buttress frame 100 extends generally axially from and radially outwardly relative to an inflow edge 110. The buttress frame 100 extends vertically, or axially, away from the inflow edge 110 and terminates along a generally horizontal outflow edge 112, generally horizontal meaning that the outflow edge may be curved or linear. Oppositely oriented ends 102 and 104 demark the width of the buttress frame 100. The outflow edge 112 provides a bottom edge of the buttress frame 100. An axial length of an inner face 108 of the buttress frame 100 between inflow and outflow edges 110 and 112 extends radially inwardly toward axis A and then radially outwardly away from the axis A. An outer face 106, oppositely oriented to the inner face 108 may likewise extend radially inwardly toward axis A and then radially outwardly away from the axis A between inflow and outflow edges 110 and 112. In other words, the buttress frame 100 is preferably curved along its width and its height. Other embodiments of the buttress frame 100 are conceivable, wherein faces 106 and 108 are not radially curved toward or away from axis A. Curvature about its width allows the buttress frame 100 to provide a sufficiently close contact surface for a properly functioning valve leaflet to coapt against without restricting blood flow through the annular opening 30 of the support frame 10.

Faces 106 and 108 are also horizontally curved, or along a length of the buttress frame 100. A radius RA measured from the axis A to either the outer face 106 or inner face 108 may be constant along a vertical plane or may change. Essentially, the buttress frame 100 may be shaped to give form to a buttress 200 or 300 that fits or conforms to a specific shape of a valve leaflet of a given patient. However, it is preferable that the buttress frame 100 is at least bilaterally symmetrical for implantation purposes. The curvature about its length ensures that the buttress frame 100 provides ample space for blood to flow through the annular opening 30 of the support frame 10 in a complete automatic repair device 400 embodiment.

The buttress frame 100 is ideally formed from a single, continuous monolithic structure composed of a wire, or wire-like material, oriented in a helix-like or uneven spiral pattern extending between ends 102 and 104. As the spiral-like structure of the buttress frame 100 does not have a constant angle between tangent lines and a fixed axis (i.e. a lateral cross-section of the helix is not a circle), the spiral-like shape of the buttress frame 100 is also referred to as an irregular helix or helix-like. Instead of a circular cross-section, the irregular helix preferably has a curved oblong shape. The overall dimensions of the curved oblong cross-sections preferably increase in succession from end 102 to a mid-point between ends 102 and 104, where dimensions are greatest, then decrease in succession from the midpoint to end 104.

The irregular helix structure of the buttress frame 100 provides the buttress frame, and buttress 200 or 300 by extension, with flexibility to be deformed from an original shape, shown in FIGS. 5-8, to either an expand form or contracted form. The buttress frame 100 is preferably made from a material with shape memory properties, such as Nitinol, that allows the buttress to be deformed from the original shape and then automatically return to the original shape.

FIG. 9 shows the buttress 200 embodiment, which includes the buttress frame 100 embodiment covered with one or more webs 205 of flexible biocompatible material that is attached over and covers the buttress frame. The biocompatible material can be a natural or biological material (e.g., a sheet of fixed and detoxified animal pericardium, dura matter) or it can be a synthetic biocompatible material (e.g., a sheet of expanded polytetrafluoroethylene, known as ePTFE). The buttress 200 has structures similar to the buttress frame 100, including an inflow edge 210 and an outflow edge 212, an inner face 208 and an outer face 206, and ends 202 and 204.

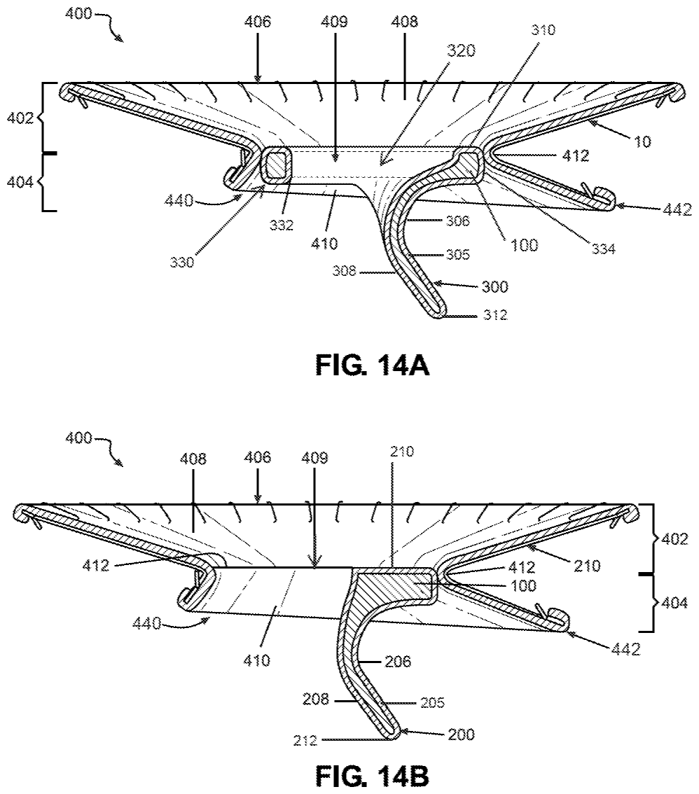

The buttress 200 is shown along a cross-section and secured as part of an embodiment of the automatic repair device 400 in FIG. 14B. The radially inner face 208 provides a surface against which a heart valve leaflet, for example, an anterior leaflet of the mitral valve, may coapt at systole. As shown in FIG. 14B, a radially outer face 206 of the buttress 200 has a generally convex or a C-shaped cross-section. The buttress frame 100 is covered by the web 205 such that, at systole, blood cannot pass back into the left atrium through the buttress 200. This may be accomplished through complete enclosure of the buttress frame 100 by the web 205, or it may be achieved through partial enclosure. The buttress frame 100 should be at least partially enclosed by the web 205 to prevent regurgitation at least to the level of a healthy, functioning heart valve.

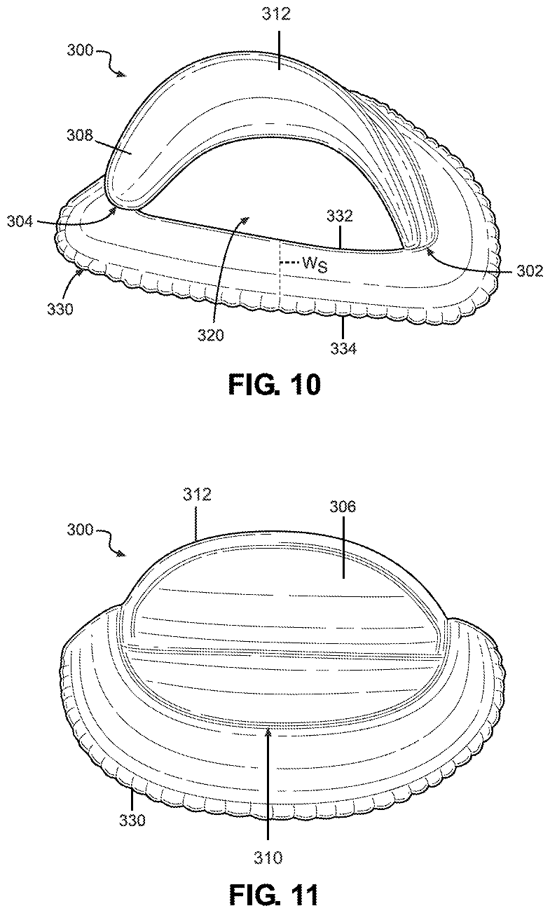

FIGS. 10 and 11 show an alternate embodiment of a buttress 300, which includes a buttress frame 100, one or more webs 305, and a flexible skirt 330. The web 305 at least partially covers the buttress frame 100, and preferably along ends 302 and 304, inflow edge 310 and outflow edge 312, and inner face 308 and outer face 306. The skirt 330 has a continuous outer edge 334 and a continuous inner edge 332, the inner edge defining an annular opening 320 of the buttress 300. A width W_{S} extends between the inner edge 332 and outer edge 334, along which the skirt 330 is preferably flexible. The inflow edge 310 of the buttress 300 is secured along, or continuous with, a partial length of the inner edge 332 of the skirt 330, such that the annular opening 320 is further defined partially by the inflow edge and the inner edge of the skirt. A cross-section of the buttress 300 as part of an embodiment of the automatic repair device 400 is shown in FIG. 14A.

Both the buttress 200 and 300, due to the buttress frame 100, are flexible, such that they are deformable from an initial natural or original shape to an expanded or contracted shape and automatically reformable to their original shape.

Further, the buttress 200 and 300 may be constructed from a solid plastic frame, or a substantially similar material, instead of the irregular helix structure frame. Such a solid frame would otherwise conform to the described shape of the buttress frame, but would not require a web 205 or 305 as a covering. The buttress 200 and 300 could be also conceivably be constructed from a dense foam covered by a web 205 or 305. Such a material would need to be dense enough to provide sufficient support for a coapting valve leaflet, but also be deformable and automatically reformable. It is preferable that any alternative material would likewise be deformable and/or compressible from a resting shape and be capable of automatically returning to the resting shape. The irregular helix frame is preferable, as volume within a catheter or similar medical delivery device is at premium. The irregular helix buttress frame 100 addresses both a volumetric concern and a deformation/reformation concern when addressing the structural concerns of delivering a properly functioning automatic repair device 400 via the most minimally invasive method.

FIG. 12 provides a side view of an embodiment of an automatic repair device body 401, which includes a support frame 10 embodiment that has been covered along at least an inner face by a web or covering 406 that is continuous along and between an inflow portion 402 and an outflow portion 404. An inner annular circumference 412 of the body 401 is defined by web 406 along the inner extent 28 of the support frame 10. The annular circumference 412 defines an annular opening 409, shown in FIG. 14A, through the body 401. An outer covering or web 430 may partially cover the support frame 10 along an outer face of the body 401, oppositely oriented to the web 406. This web 430 may partially cover the outer face of the support frame 10 along a portion corresponding to the inner extent 28. Stitching 432 or similar means of fastening the webbing 430 and 406 together or to the support frame 10 may be present along the annular circumference 412 and inner extent 28. While the web 406 and 430 are each preferably one piece, the web may include one or more webs. The web 406 is preferably a flexible biocompatible material that is attached over and covers the respective inflow and outflow portions 402 and 404. The biocompatible material can be a natural or biological material (e.g., a sheet of fixed and detoxified animal pericardium, dura matter) or it can be a synthetic biocompatible material (e.g., a sheet of expanded polytetrafluoroethylene, known as e-PTFE). Preferably, the web 406 partially covers an outer surface of the support frame 10 along at least the distal ends 20 and 22, such that suture can be passed through two layers of the web and the eye 40, 42 of each distal end to secure the web to the support frame.

Angle Θ_{N} is the natural or original angle between the inflow portion 402 and outflow portion 404 while the support frame 10, and body 401 by extension, is in the resting configuration. The angle between the inflow portion 402 and outflow portion 404 may vary as the body 401 is compressed and relaxed between the resting configuration and a deformed configuration. However, the angle automatically returns to the angle Θ_{N} once outside forces no longer prevent the body 401 from returning to the resting configuration. The angle Θ_{N} is measured between the inflow portion 402 and outflow portion 404, similar to angle Θ_{F} of the support frame 10. In most embodiments, the angle Θ_{N} should be identical to the angle Θ_{F}. Preferably, the angle Θ_{N} is 20-45° to conform to the corresponding heart valve annulus. Alternatively, the angle Θ_{N} can be adjusted or manufactured to match a corresponding angle around the heart valve annulus to ensure a secure implantation of the automatic repair device 400.

The preferred embodiment of the automatic repair device 400 includes all elements of the previous body 401, but additionally includes a buttress 200 or 300 secured around the annular opening 409. In FIG. 13, the buttress 300 is included in the automatic repair device 400.

FIG. 14A provides a cross-sectional view of the automatic repair device 400 with the buttress 300 incorporated with the body 401, as shown in FIG. 13. In this embodiment, the skirt 330 of the buttress 300 is secured in the annular opening 409 along the annular circumference 412 of the body 401, with the buttress extending from the annular circumference toward and past the outflow portion 404. The skirt 330 is secured and sealed at least to the web 406 along the annular circumference 412, and preferably also to one or more inflow support features 16 and outflow support features 18 of the support frame 10, such that all blood flow through the automatic repair device 400 will be directed through the annular opening 320. In other words, the buttress 300 should be sealed to the body 401, such that blood can only flow through opening 320, and not between the outer edge 334 and web 406 along the annular opening 409. An inflow surface 408 and outflow surface 410, both formed by the one or more webs 406 covering an inner face of the support frame 10, ensure that there is no lateral blood leakage through the inflow and outflow portions 402 and 404, or at least sufficiently minimal lateral leakage.

Another embodiment of the automatic repair device 400 may include the buttress 200, instead of the buttress 300, as shown in FIG. 14B. The buttress 200 extends from the circumference 412 toward and past the outflow portion 404. In this embodiment, the buttress 200 is secured to the annular circumference 412 along the inflow edge 210, such that blood cannot flow between the rear surface 206 and the outflow surface 410. Instead, blood is directed to flow through the annular opening 409 defined by the annular circumference 412 and the contact surface 208 of the buttress 200. The buttress 200 may be attached to the body 401 via the web 205 being attached to web 405. Additionally, the buttress frame 100 may be attached to the support frame 10 by wire, suture material, or the like.

In both embodiments shown in FIGS. 14A-14B, the buttress frame 100 allows the buttress 200 and 300 to deform and reform to the resting configuration along with the rest of the prosthesis, thereby allowing the entire automatic repair device 400, to be deformed when loaded in a catheter and completely be reformed after being deposited in the valve annulus.

FIG. 15 illustrates another embodiment of a support frame 600, which may be incorporated into an embodiment of the automatic repair device 400 instead of support frame 10. The support frame 600 is similar to support frame 10, but is formed of a separate inflow support frame 602 and an outflow support frame 604 instead of a single continuous monolithic structure. As an example, each of the respective inflow and outflow support frames 602 and 604 can be formed from a single loop or circular wire material, such as from materials commonly used to make stents, which is then bent and formed without being cut or otherwise separated. Alternatively, the two or more pieces of material may be attached together as known in the art to form a continuous monolithic structure for each of the respective inflow and outflow support frames 602 and 604.

Each support frame 602 and 604 has a substantially frusto-conical configuration, with a respective opening 603 and 605 corresponding to a frustum that extends there through to what would be a base of each inflow and outflow support frame. The diameter of each opening 603 and 605 is the same in a normal, resting position, so that the openings 603 and 605 together may help form the annular opening 409 of the automatic repair device when fully assembled and covered by the web 406.

Each support frame 602 and 604 has a generally sinusoidal or zig-zag sidewall pattern formed through individual inflow support arms 607 and outflow support arms 609, extending axially and radially outwardly from a frustum end adjacent to respective opening 603 and 605 to an opposing base end. A pair of inflow support arms 607 extending axially and radially outwardly in opposite directions, but toward each other, meet at a distal end 606 to form an inflow support feature 611 also extending axially and radially outwardly from the opening 603. The inflow support frame 602 has a plurality of inflow support features 611. Likewise, two outflow support arms 609 extending axially and radially outwardly in opposite directions, but toward each other, meet at a distal end 608 to form an outflow support feature 613 also extending axially and radially outwardly from the opening 605. Relative to their respective support frame 602 and 604, the support features 611 and 613 are continuous with adjacent support features 611 and 613, respectively, but do not alternate in opposite directions, i.e. upwardly or downwardly, like with support frame 10. Each distal end 606 of the inflow support frame 602 extends in the same direction, which is away from the frustum or opening 603. Likewise, each distal end 608 of the outflow support frame 604 extends in the same direction, which is away from the frustum or opening 605. In other words, inflow support frame 602 is composed only of a plurality of inflow support features 611, each inflow support feature adjacent to other inflow support features. The outflow support frame 604 is composed only of a plurality of outflow support features 613, each outflow support feature adjacent to other outflow support features.

In this manner, the zig-zag pattern of each support frame 602 and 604 is formed by respective support arms 607 and 609. For support frame 10, the zig-zag pattern is created by alternating inflow and outflow support features 16 and 18.

Adjacent distal ends 606 are continuous with each other along support arms 607 meeting at an inner end 615. Each support feature 607 extends between a distal end 606 and an inner end 615. Each inner end 615 is positioned adjacent to the opening 603. The zig-zag sidewall pattern is therefore more particularly formed by support arms 607 alternating between distal ends 606 and inner ends 615. Each distal end 606 and inner end 615 preferably has an eye or hole 624 for receiving and securing string, line, cord, or similar suture material there through. A cord 618 may be inserted through the eye 624 of each inner end 615 to form a complete loop, which further defines opening 603. The cord 618 can be used to reduce the opening 603 to a desired diameter to selectively provide the radially inner dimension of the inflow support frame 602. Alternatively, the cord 618 may not be used to reduce or influence the shape of the opening 603, such as when the inflow support frame is constructed to provide the desired radially inner dimension of the opening, and may instead be utilized to provide a structure upon which other independent structures may be attached to the inflow support frame 602 through sutures, such as a web 405, buttress 200 or 300, or the outflow support structure 604.

Likewise adjacent distal ends 608 are continuous with each other along support arms 609 meeting at an inner end 617. Each support arm 609 extends between a distal end 608 and an inner end 617. Each inner end 617 is positioned adjacent to the opening 605. The zig-zag sidewall pattern of support frame 604 is therefore more particularly formed by support features 609 alternating between distal ends 608 and inner ends 617. Each distal end 608 and inner end 617 preferably has an eye or hole 626 for receiving and securing string, line, cord, or similar suture material there through. A cord 620 may be inserted through the eye 626 of each inner end 617 to form a complete loop, which further defines opening 605. The cord 620 can be used to reduce the opening 605 to a desired diameter to selectively provide the radially inner dimension of the outflow support frame 604. Alternatively, the cord 620 may be utilized to provide a structure upon which other independent structures may be attached to the outflow support frame 604 through sutures, such as a web, buttress, or the inflow support structure 602.

Those skilled in the art will appreciate other ways to achieve desired shape and configuration for each of the support frames 602 and 604, which can include alterations in shape and dimensions of individual elements of the support frames or using other means for reducing one or more of the ends. The inflow support frame 602 and outflow support frame 604 should have a similarly sized and shaped opening 603 and 605 to facilitate conjoining the support frames and together along the openings, and cords 618 and 620 when present, when forming a complete automatic repair body 401 and device 400.

Like support frame 10, the length of the outflow support features 613 is overall shorter than inflow support features 611. The inflow portion length L_{I} is measurable along any inflow support feature 611, and is defined between the distal end 606 of the inflow support feature and a midpoint between the inner end 615 on either side of the distal end of the inflow support feature along a circumference of opening 603. This circumference is determined by the inner ends 615 of the inflow support frame 602. Alternatively, the length L_{I} can be measured from a midpoint between the inner ends 615 along the cord 618, if present, for ease of reference. The length L_{I} of any inflow support feature 611 is longer than any length L_{O} of an outflow support feature 613, which is defined between the distal end 608 of an outflow support feature 613 and a midpoint between the inner end 617 on either side of the distal end of the inflow support feature along a circumference of opening 605. Alternatively, the length L_{O} can be measured from a midpoint between the inner ends 617 along the cord 620, if present, for ease of reference. Measurements of length L_{I} and L_{O} should be consistently measured using relative points of reference along support frames 602 and 604.

The outflow support frame 604 is overall shorter than the inflow support frame 602 when comparing lengths of support features. Preferably, the longest outflow support feature 613 length L_{O} is two-thirds of the inflow support feature 611 length L_{I}. However, other embodiments of the support frame 600 may have a different proportion where L_{O}<L_{I}.

Each inflow support feature 611 of a support frame 602 shares the same length L_{I}, although the value of L_{I} may vary between different embodiments of the support frame. This creates an inflow portion 12 that is uniform in shape and size. However, the length L_{O} varies between one or more outflow support features 613 of the outflow support frame 604. Like support frame 10, shorter outflow support features 613 are located together along one side of the support frame 604, instead of being interspersed among longer outflow support features. The outflow support features 613 may gradually change in length L_{O} along adjacent outflow support features or there may be two groups of outflow support features, each group having its own value for length L_{O}, to create a group of short outflow support features and a group of long outflow support features. Outflow support features 613 may be oriented similarly or differently to outflow support features 18 of support frame embodiments 10 shown in FIGS. 2A-3B.

However, unlike support frame 10, support frame 600 does not have to have an identical amount of inflow support features 611 and outflow support features 613 between the support frames 602 and 604, as the inflow support frame and outflow support frame are not continuous with each other. For example, support frame 602 may have six inflow support features 611, while support frame 604 has eight outflow support features 613. In another embodiment, support frame 602 may have seven inflow support features 611, while support frame 604 has six outflow support features 613. Of course, support frames 602 and 604 may also have an identical amount of inflow support features 611 and outflow support features 613, respectively.

When being connected together to form support frame 600, the support frames 602 and 604 may be secured through suture, wire, or similar flexible material fastening corresponding holes 624 and 626 together. In this manner each inner end 615 is secured to a corresponding inner end 617. This manner of securing the support frames together works best when there are an equal number of inflow support features 611 and outflow support features 613. Not all corresponding inner ends 615 and 617 need be connected together, although it is preferable. Additionally, the support frames 602 and 604 may be connected together along cords 618 and 620. A wire, suture, or string may be looped around cords 618 and 620, and optionally through holes 624 and 626, to connect the support frames 602 and 604 together.

Once support frames 602 and 604 are secured together, the support frame 600 provides a generally hourglass shape in its natural or original position in which it has a substantially V-shaped cross-sectional configuration along a longitudinal cross-section taken radially outwardly through one side of the fixation support in its resting configuration. This substantially V-shaped cross-sectional configuration can include a V-shaped configuration, a U-shaped configuration, or a configuration between two such shapes.

The support frame 600, once support frames 602 and 604 are secured together, has an angle Θ_{F} measured between the inflow and outflow support frames and is preferably between 20-45° (degrees), inclusive, to properly conform to a valve annulus. However, it is conceivable that the angle Θ_{F} could be outside of this range to accommodate specific patient conditions. The angle Θ_{F} is preferably consistent about a circumference of the support frame 600 for a given embodiment, but may be different between embodiments or to conform to anatomy of a specific patient.

Like support frame 10, the support frame 600, does not apply unwanted lateral pressure to certain cardiac structures when inserted within a valve annulus, such as the mitral valve annulus. As discussed, outward lateral pressure around the mitral valve, especially on the ventricular side of the mitral valve can lead to death by occlusion of the coronary artery. The support frame 600 allows the heart valve to be secured in the mitral valve using axial pressure, which involves the annulus being clamped between the inflow support frame 602 and outflow support frame 604 via the inflow support features 611 and outflow support features 613, respectively. Such vertical forces do not present the same health risks as the outward horizontal forces previously discussed. Further, the uneven outflow support frame 604 allows the support frame 600 to be positioned such that a shorter area 630 of the outflow support frame is oriented toward a properly-functioning valve leaflet. The properly-functioning leaflet can freely move toward central axis C of the support frame 600 via the shorter area 630 to partially close the annular openings 603 and 605, in conjunction with a buttress 200 or 300. A longer area 632 of the outflow portion 604 can be positioned over a non-functioning leaflet of the valve to be repaired through effective replacement.

Each of the inflow and outflow support frame 602 and 604 can also include spikes similar to support frame 10. For the inflow support frame 602, one or more spikes 610 may extend outwardly from each distal end 606, and one or more spikes 614 may extend outwardly from each inner end 615. For the outflow support frame 604, one or more spikes 612 may extend outwardly from each distal end 608, and one or more spikes 616 may extend outwardly from each inner end 617. Various numbers and configurations of the spikes 610, 612, 614, and 616 can be implemented, such as single spike or more than two spikes at some or all of the ends. Additionally, ends of all or some spikes 610, 612, 614, and 616 can have tapered or sharpened and/or barbed tips to facilitate gripping surrounding tissue when implanted.

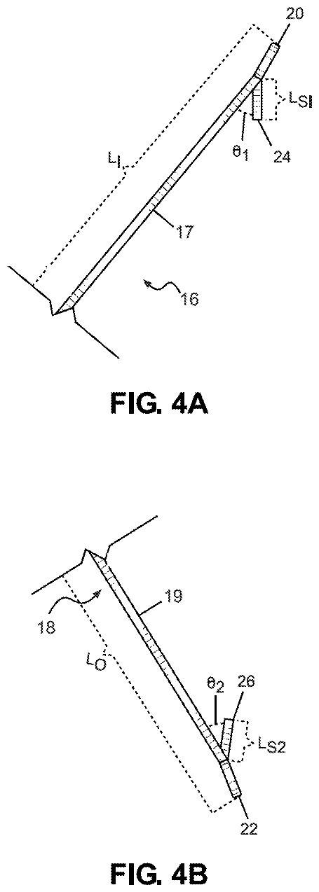

Preferably, each inflow spike 610 and 614 has an angle Θ₁ of 30° relative to the corresponding inflow support arm 611 or inner end 615 to which it is attached. Further, each inflow spike 610 and 614 has a length L_{SI}. Each outflow spike 612 and 616 preferably has an angle Θ₂ of 45° relative to the outflow support arm 613 or inner end 617 to which it is attached, and a length L_{SO} which is 2 mm (millimeters) longer, or equals L_{S1}+2 mm, than the inflow spikes 610 and 614. The length of spikes 610, 612, 614, and 616 may otherwise vary between embodiments to a desired length. These lengths and angles are similar to those of spikes 24 and 26 shown in FIGS. 4A and 4B

As described herein, the automatic repair device 400 embodiments can be implanted through a low invasive procedure, which may include no cardio pulmonary bypass or may be implemented with a reduced amount of cardio pulmonary bypass relative to other mitral valve replacement procedures. Additionally, when implanted, the automatic repair device 400 can be implanted without removing the patient's native mitral valve, as shown. However, the prosthesis can also be implanted if the patient's value is partially removed.

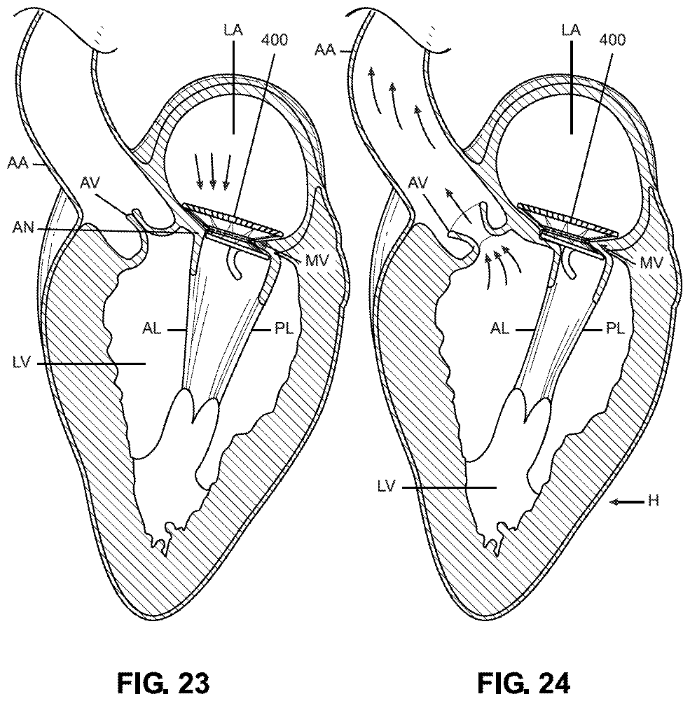

Embodiments of a method for implanting an automatic repair device 400 are included herein. For consistency of explanation and not by way of limitation, the methods will be described with respect to the example embodiment of the automatic repair device 400 of FIG. 14A and implanted at the annulus AN of a mitral valve MV as depicted with respect to FIGS. 23-26. It will be appreciated that other configurations and combinations of the automatic repair device 400 embodiments shown and described herein can be implanted according to the methods described herein. Additionally, while implantation methods will be described with respect to implanting the automatic repair device 400 at the mitral position, it should be understood that these same methods can be adapted for implantation at the annulus of another cardiac valve, including the aortic valve. Thus, the devices 400 shown and described herein are applicable for implantation at either atrio-ventricular position, and at other cardiac valve sites. Further, additional methods of implanting embodiments of the automatic repair device may be used, including known methods of implantation involving open-heart surgery, e.g. non-percutaneous methods, or other methods of percutaneous implantation that allow for contraction and subsequent expansion of the automatic repair device in the valve annulus.

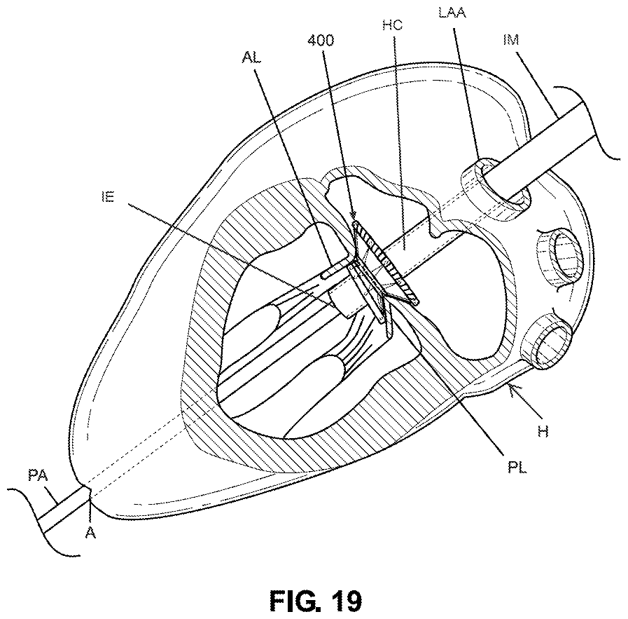

Prior to implanting the automatic repair device 400 at a desired implantation site, the automatic repair device is inserted within an implanter IM, such that the device has the reduced cross-sectional dimension relative to the expanded or original cross-sectional dimension of the device. The implanter IM can be similar to the type shown and described with respect to FIG. 19 of U.S. patent application Ser. No. 10/266,380, filed on Oct. 8, 2002, and entitled HEART VALVE PROSTHESIS AND SUTURELESS IMPLANTATION OF A HEART VALVE. The implanter in the referenced patent application provides a substantially linear barrel, which can have a flexible or bendable tip to facilitate direct implantation through the heart to the desired implantation site. The support frame 10 or 600 of the automatic repair device 400 is preferably made out of a flexible material with memory shape properties, which allows the device to be compressed enough to be loaded into other types of known implanters, including catheters and trocars, for performing the methods described herein. Prior to this invention, the materials used to make prostheses resulted in mitral valve prostheses that were too big to load into smaller diameter loaders without damaging the prostheses and rendering them inoperable. A 6 mm diameter barrel B is preferably the smallest width barrel to use in order to both accommodate the compressed automatic repair device 400 and fit through the necessary annulus and other structures of the patient's heart and body. However, a smaller barrel B is conceivable if materials allow for smaller dimensions of a compressed device 400 that is still structurally safe for the patient.

Returning to the example of FIG. 16, which shows a trocar device as the implanter IM, before inserting the implanter into a heart H, an opening is created in the heart to provide a substantially direct path to a valve annulus AN in the heart. As used herein, the term “substantially” as modifier for “direct path” is intended to convey that the opening is intended to provide a line-of-sight path from the opening to the implantation site, although some deviation might exist. Such deviation can be compensated, for instance, by employing a bendable implanter IM that can be inelastically deformed to a shape to facilitate implantation at the site or by deforming the heart H manually during the procedure to provide the corresponding path along which the implanter can traverse. This is to be contrasted with percutaneous implantation procedures that are performed through femoral vein, for example.

As a further example, a mattress suture, or other type of purse string suture can be applied at location in the patient's heart through which the implanter IM is to be inserted. In the example of FIG. 16, the location comprises the patient's heart muscle located at an apex AP of the heart H. Two ends of the purse string suture extend from the apex AP tissue can be tightened around the implanter IM to mitigate blood loss. Consequently, cardiopulmonary bypass is not required. However, it is to be understood that in certain situations, some bypass may be necessary, although usually for a much shorter period of time than with conventional procedures. The collapsed automatic repair device 400 can be pre-loaded in an implantation end IE of the implanter IM, or passed through a hollow cavity HC of the implanter from outside of the body and to the implantation end for insertion at the valve annulus AN.

The automatic repair device 400, in its reduced cross-sectional dimension, can have a diameter of 5 mm or more and expand to a 24-35 mm diameter at the implantation site. Those skilled in the art will appreciate that the expanded valve dimensions (e.g., ≥24 mm) are typically not suitable for percutaneous implantation procedures. However, such sizes of devices are deemed appropriate and sometimes necessary for replacement of the mitral valve. Additionally, many existing manufactured pericardial valves designed for minimally invasive percutaneous implantation are not effective at such large sizes and/or are not capable of operating under the hemodynamic conditions that typically exist for the mitral position. The multiple embodiments of the automatic repair device 400 provided herein are capable of insertion via catheters and trocars having diameters between 5-10 mm, expansion to natural shape of 24-35 mm, and structural integrity to operate under hemodynamic conditions.

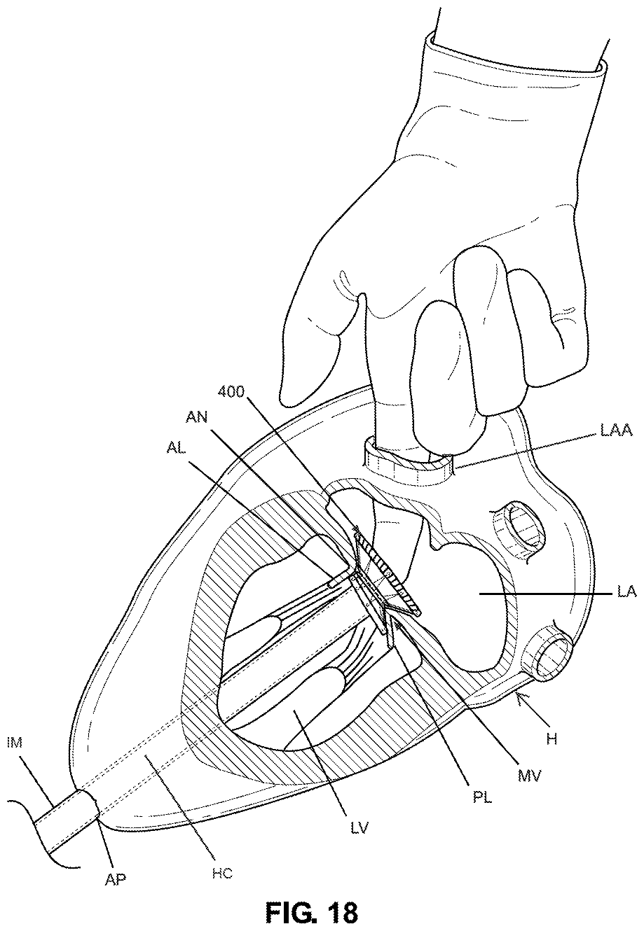

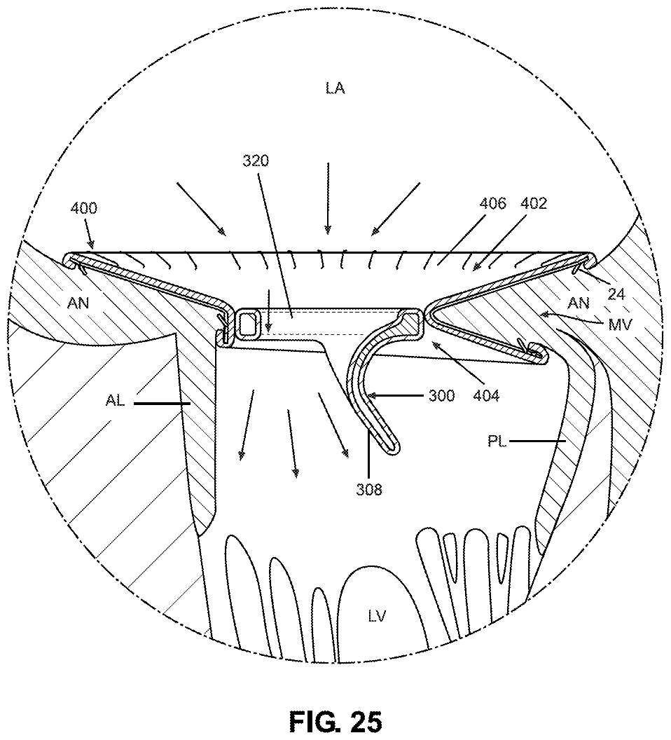

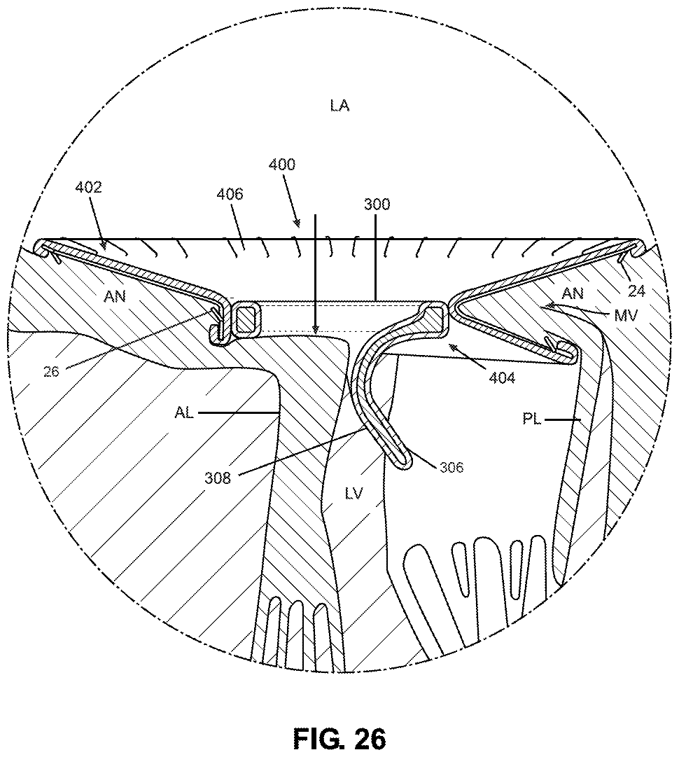

After the implanter IM of FIG. 16 is inserted through the apex AP and the implantation end IE is in proper position near the valve annulus AN, the insertion of the automatic repair device 400 can be guided by a surgeon's finger, as shown in FIG. 18, or other instrument that is introduced via the left atrial appendage LAA. A purse string or mattress suture can be applied around the left atrial appendage LAA to mitigate blood loss. The surgeon's finger can locate the patient's native mitral valve MV and associated annulus AN to help position and guide the implantation end IE of the implanter IM to the desired implantation site. Once at the desired site, the automatic repair device 400 can be discharged from the implantation end IE of the implanter IM and the implanter can be withdrawn from the heart H. The finger (or other instrument) can also be used to help guide the device to ensure its proper fixation and implantation at the appropriate position at the mitral annulus AN, such as shown in FIGS. 25 and 26.

After the automatic repair device 400 has been expanded to its expanded cross-sectional dimension, which may be performed automatically by reformation of the device to its original size or by manual means such as the balloon catheter or other mechanism for expanding the device, the device is fixed at the desired position. Optionally, one or more sutures can be applied externally through the heart H to help anchor the automatic repair device 400 at the desired implantation position. Alternatively, a trocar or other device can be inserted through the left atrial appendage or otherwise to provide a suture or other means for further securing the automatic repair device 400 at such position.

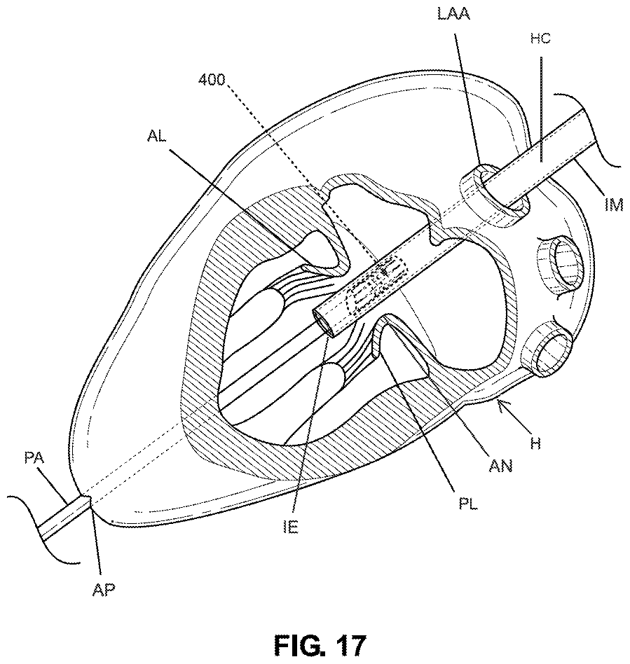

FIG. 17 depicts an alternate implantation method in which the implanter IM and automatic repair device 400 are inserted through the left atrial appendage LAA for direct implantation at the mitral annulus AN. The steps up to insertion of the barrel into the left atrial appendage LAA are similar to those described with respect to FIG. 16. Briefly, the chest is opened, a purse string suture applied about the left atrial appendage LAA and the implantation end IE of the implanter IM is inserted through the opening in the atrial appendage to pass through the left atrium LA to the valve annulus AN. The purse string can be tightened about the implanter IM to mitigate blood loss, thereby forgoing cardio-pulmonary bypass unless needed in emergency situations.

In order to facilitate proper positioning of the automatic repair device 400, a positioning apparatus PA (e.g., a dilator or umbrella or other structure) can be inserted through the heart muscle, such as the apex AP of the heart H, and positioned to a desired location. A purse string suture can be employed at the apex AP and tightened around the apparatus PA to control bleeding. The placement of the positioning apparatus PA can be guided by fluoroscopy or other imaging modalities. By way of example, positioning apparatus PA can include an umbrella-type distal end that is attached to a shaft. The distal end can be inserted in a closed condition through the apex AP to a desired position the patient's heart valve and expanded to an open condition. In the open condition, the distal surface of the opened umbrella provides a back stop against which the implantation end IE of the implanter IM or automatic repair device 400 can engage for defining an implantation position. For instance, once the implantation end IE of the implanter IM engages the distal end, which can be felt or otherwise perceived by the surgeon, the automatic repair device 400 automatic repair device 400 can be discharged from the implantation end IE at the mitral valve annulus AN. When expanded, the inflow and outflow portions 402 and 404 can receive tissue at the mitral annulus AN and thereby hold the automatic repair device 400 at a fixed axial position relative to the mitral annulus AN, as shown in FIG. 19.

Additionally, the described implantation can be performed percutaneously without opening the patient's chest cavity. The implantation end IE of the implanter IM can be inserted through the femoral vein using a transseptal access to the left atrium LA or through the apex AP in a transapical approach.

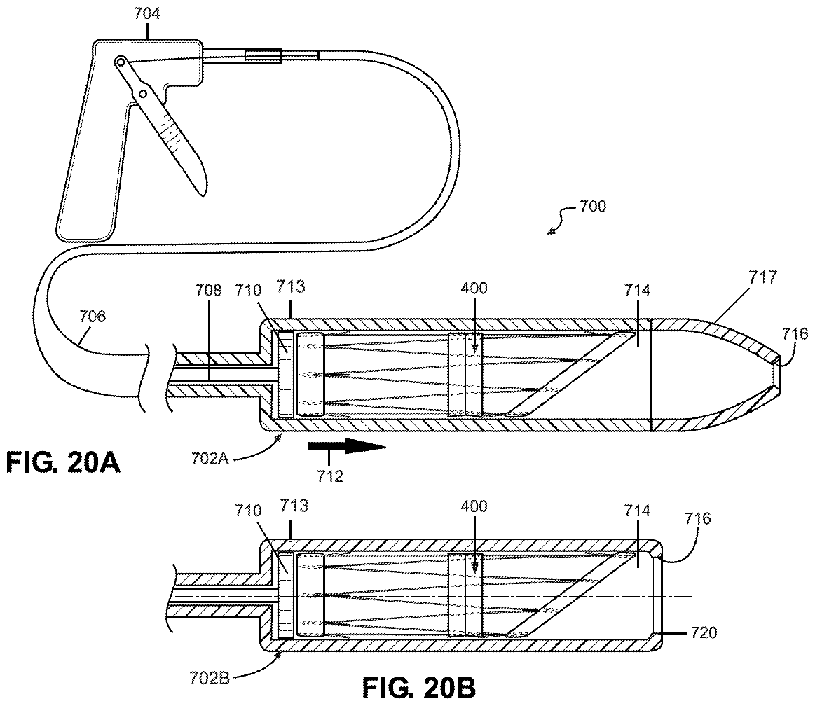

FIGS. 20A and 20B depict different embodiments of another apparatus 700 that can be used as the implanter IM for delivering the automatic repair device to the valve annulus AN. The apparatus 700 is in the form of a catheter device having a trigger element 704 connected to an enclosure 702A by a hollow connecting element 706. An extendable member 708 is connected to and operable by the trigger mechanism 704. The extendable member runs through the connecting element 706 and terminates at a plunger element 710 housed within the enclosure 702A. Operation of the trigger mechanism causes the plunger element 710 to move within a hollow cavity 714 of the enclosure 702A, defined the by enclosure body 713, and in a direction 712 toward an opening 716 along an end of the enclosure opposite to the connecting element 706. Deactivating the trigger mechanism 704 allows the plunger element to move back in a direction opposite to direction 712. Embodiments of the automatic repair device are containable within the hollow cavity 714 in their collapsed or condensed configuration. The plunger element 710 is configured to push the automatic repair device out of the enclosure 702A after the enclosure has been inserted into the heart at the valve annulus AN. FIG. 20B shows an enclosure 702B having a differently sized opening 716 that the opening 716 of enclosure 702A. Further, enclosure 702A has a tapered or conical end 717, whereas enclosure 702B instead has a flanged surface along the opening. In both cases, the enclosures 702A and 702B are shaped to keep the automatic repair device 400 within the hollow cavity 714 until the plunger element 710 is activated to push the device out through the opening 716.

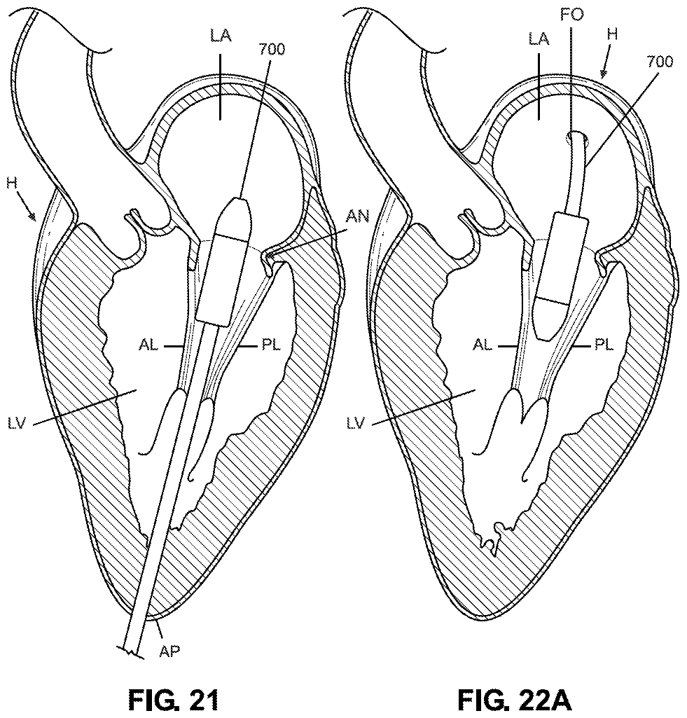

Similar to the implantation method shown in FIG. 16, the apparatus 700 can be inserted through the apex AP until is in proper position near the valve annulus AN, as shown in FIG. 21. A purse string or mattress suture can be applied at the apex AP and around the connecting element 706 to mitigate blood loss. Once at the desired site, the automatic repair device 400 can be discharged from the enclosure 702A and the implanter can be withdrawn from the heart H. A finger, or other instrument, through the left atrial appendage LAA can also be used help guide the device to ensure its proper fixation and implantation at the appropriate position at the mitral annulus AN. In this configuration, the automatic repair device 400 would be oppositely positioned within the enclosure body 713, compared to its position shown in FIGS. 20A and 20B, such that the inflow portion is positioned toward the opening 716.

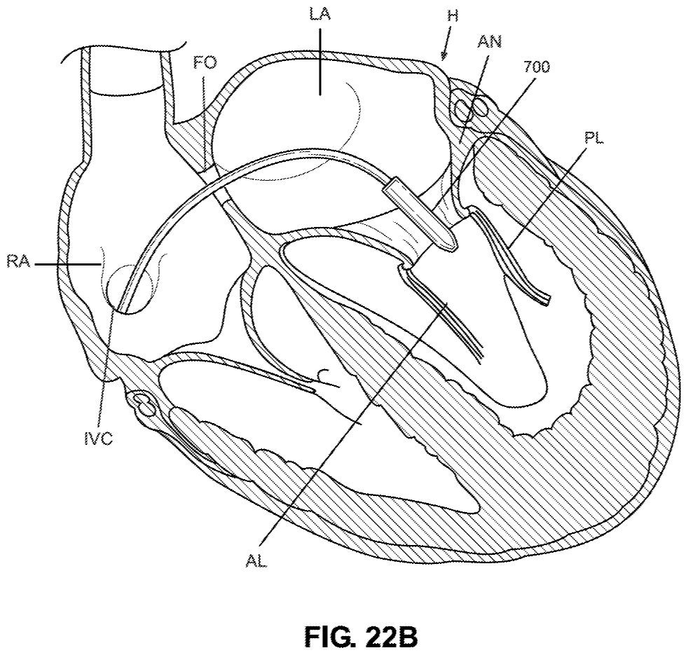

FIGS. 22A and 22B show insertion of the apparatus through the fossa ovalis FO that separates the right and left atrium. This method of insertion can begin by making an insertion along the patient's thigh and femoral vein. The apparatus 700 is then inserted into the femoral vein and fed up towards and into the inferior vena cava IVC. The apparatus is then passed through the inferior vena cava IVC and into the right atrium RA. The apparatus 700 can then be pushed through an incision made along the fossa ovalis FO and into the left atrium LA, where the apparatus can then be positioned within the valve annulus AN.

These methods of implantation can be performed percutaneously without the need to invasively open the patient's chest. X-rays and other imaging systems can help guide the surgeon or operator to properly guide the implanter IM or apparatus 700 through the patient's body and properly insert the automatic repair device 400 in and around the valve annulus AN. The longer spikes 26 of the outflow portion are advantageous in this regard, as they are distinguishable in imaging systems and can therefore be used as guides in combination with such systems to properly implant the device.

FIGS. 23 and 24 illustrate a heart H with an automatic repair device 400 implanted in an annulus AN of a heart valve, in this case a mitral valve MV. FIG. 23 shows the heart H during diastole. Blood, indicated with arrows, flows from the left atrium LA through opening 320 in the automatic repair device 400 and into the left ventricle LV. An aortic valve AV is closed, preventing blood flow from the left ventricle LV to the aorta AA. An anterior leaflet AL and a posterior leaflet PL of the mitral valve MV are relaxed to allow blood to pass through the annulus AN of the mitral valve.

During systole, shown in FIG. 24, the left ventricle LV contracts to expel blood, again, shown in arrows. The anterior leaflet AL and posterior leaflet PL, in a healthy person, would contract and move toward each other in the annulus AN of the mitral valve MV to block blood flow through between the left atrium LA and left ventricle LV. However, in about 80 percent of patients with mitral valve complications, the posterior leaflet PL is defective. The automatic repair device 400 is inserted in and around the annulus AN such that the buttress 200 or 300 extends downwardly from the annulus into the left ventricle LV. This provides a contact surface for the anterior leaflet AL to contact, or coapt against, during systole to prevent blood flow between the left atrium LA and left ventricle LV. In turn, the aortic valve AV is opened and blood flows from the left ventricle LV through the aorta AA.

FIG. 25 provides an enlarged view of the automatic repair device 400 inserted within and around the annulus AN of a heart valve HV during diastole, as shown in FIG. 23. In this illustration, the automatic repair device 400 of FIG. 14A is provided for reference. The device 400 is implanted in the annulus AN of the mitral valve MV. The inflow portion 402 of the device 400 extends within the left atrium LA, while the outflow portion 404 extends within the left ventricle LV. A shorter side 440 of the outflow portion 404 is oriented toward the anterior leaflet AL, while longer side 442 of the outflow portion extends over the defective posterior leaflet PL. The buttress 300 extends downwardly into the left ventricle LV, with the inner face 308 preferably oriented toward the properly functioning leaflet, in this case the anterior leaflet AL. During diastole, blood flows from the left atrium LA through the automatic repair device 400 via the annular opening 320 of the buttress 300 and into the left ventricle LV.

FIG. 26 shows the automatic repair device 400 interacting with the anterior leaflet AL during systole. The anterior leaflet AL contracts upwardly and toward the inner face 308 of the buttress 300, which in turn acts as the posterior leaflet to prevent blood flow through the annular opening 320 of the buttress 300 and in turn through the mitral valve MV. As it is important to minimize blood leakage from the left atrium LA to the left ventricle LV during systole and diastole, the inflow portion 402 and outflow portion 404 are designed to conform to the shape of the annulus AN to prevent leakage between the annulus and the automatic repair device 400. Likewise, the buttress 300 is secured to the web 406 of the automatic repair device 400 so that blood flow is directed only through the annular opening 320 during diastole. The inflow surface 408 directs blood from the left atrium LA, through the annular opening 320, and into the left ventricle LV. The outflow surface 410 helps to prevent backflow of blood from the left ventricle LV to the left atrium LA. Spikes 24 and 26 of the support frame 10 of the automatic repair device 400 help secure the device in place by shallowly penetrating cardiac tissue around the annulus AN. The oppositely oriented spikes 24 and 26 are especially helpful in ensuring that the prosthesis does not move up, down, or rotate after implantation, given that the anterior leaflet contacts against the automatic repair device 400 during diastole and may otherwise jostle or displace the device.

I claim:

1. An automatic repair device comprising:
a fixation support member having
an annular support frame formed from a plurality of support features, the plurality of support features including inflow support features and outflow support features, the inflow support features extending in an inflow direction from a circumferential path and the outflow support features extending in an outflow direction from the circumferential path, and
a support member web at least partially encasing the annular support frame to provide at least an inflow contact surface and an outflow contact surface,
wherein the outflow support features are shorter than the inflow support features;
a buttress having
a buttress support frame, and
a buttress web at least partially encasing the buttress to form a surface against which at least one leaflet of a heart valve may engage the buttress,
wherein the buttress is secured to the annular support frame along the circumferential path such that the buttress extends in the outflow direction;
wherein the fixation support member and buttress are flexible between a compressed configuration and an expanded configuration.

2. The automatic repair device of claim 1, wherein the inflow support features and outflow support features extending from the circumferential path provide the automatic repair device with one of a U-shaped or V-shaped cross-sectional configuration for a longitudinal cross-section taken axially through the prosthesis in its expanded cross-sectional dimension.

3. The automatic repair device of claim 1, wherein a spike extends from each inflow support feature and along an outer attachment surface of the fixation support member.

4. The automatic repair device of claim 3, wherein an angle between the spike of each inflow support feature and a corresponding inflow support feature is 30°.

5. The automatic repair device of claim 1, wherein a spike extends from each outflow support feature along an outer attachment surface of the fixation support member.

6. The automatic repair device of claim 5, wherein an angle between the spike of each outflow support feature and a corresponding outflow support feature is 45°.

7. The automatic repair device of claim 1, wherein at least one inflow spike extends from each inflow support feature and along an outer attachment surface of the fixation support member, and at least one outflow spike extends from each outflow support feature along the outer attachment surface of the fixation support member.

8. The automatic repair device of claim 7, wherein the at least one outflow spike is at least 2 mm (millimeters) longer than the at least one inflow spike.

9. The automatic repair device of claim 1, wherein the outflow support features are at least two-thirds shorter than the inflow support features.

10. The automatic repair device of claim 1, wherein the annular support frame and the buttress support frame are constructed from a memory shape material.

11. The automatic repair device of claim 10, wherein the memory shape material is Nitinol.

12. The automatic repair device of claim 1, wherein the automatic repair device automatically resumes a fixed shape when transitioning from the compressed configuration to the expanded configuration

13. The automatic repair device of claim 1, wherein the annular support frame is a single, continuous monolithic structure.

14. An automatic repair device comprising:
a buttress apparatus having a generally arcuate base portion and a buttress, the buttress apparatus having an inflow end and an outflow end, the buttress extending generally radially inwardly and axially along the outflow end relative to the base portion so as to permit substantially bi-directional flow of blood axially relative to the buttress apparatus, the buttress having a surface dimensioned and configured to be engaged by at least one leaflet of a heart valve when the automatic repair device is implanted at the heart valve, whereby when the automatic repair device is implanted at the heart valve, movement of the at least one leaflet of the heart valve relative to the surface of the buttress provides substantially unidirectional flow of blood relative to the apparatus; and
a fixation support member secured along a circumference of the arcuate base portion of the buttress apparatus, the fixation support member comprising inflow and outflow portions, the inflow portion of the fixation support member extending from a radially inner contact surface of the fixation support member radially outwardly and axially in a direction of the inflow end of the buttress apparatus upon deployment of the automatic repair device, the outflow portion of the fixation support member extending from the radially inner contact surface radially outwardly and axially in a direction away from the inflow portion of the fixation support member upon deployment of the automatic repair device, the buttress apparatus and the fixation support member being deformable between a reduced cross-sectional dimension and an expanded cross-sectional dimension thereof when the automatic repair device is deployed, whereby implantation of the automatic repair device is facilitated,
wherein the fixation support member comprises an annular frame support formed from a plurality of support features, the support features alternate extending between the inflow and outflow directions along a circumferential path corresponding to the radially inner contact surface, the support features being interconnected at first junctures at an axial extent of the inflow portion and being interconnected at second junctures at an axial extent of the outflow portion, each support feature being directly connected to only two other support features such that the support features collectively extend along the entire axial length of the fixation support member, and
wherein the support features extending in the outflow direction are shorter than the support features extending in the inflow direction, such that a diameter of the outflow portion of the fixation support member is smaller than the diameter of inflow portion of the fixation support member.

15. An automatic repair device, comprising:
a fixation support member having an inflow portion and an outflow portion,
the inflow portion having an outer inflow circumference and an inner inflow circumference with an inflow contact surface defined there between and extending radially outwardly and axially from the inner inflow circumference to the outer inflow circumference when the automatic repair device is in a deployed configuration,
the outflow portion having an outer outflow circumference and an inner outflow circumference with an outflow contact surface defined there between and extending radially outwardly and axially from the inner outflow circumference to the outer outflow circumference when the automatic repair device is in a deployed configuration,
wherein the inflow portion and the outflow portion are coaxially secured along the inner inflow circumference and the inner outflow circumference to form a circumferential path between the inflow portion and outflow portion, such that the fixation support member has a generally hyperboloidal shape in the deployed configuration, and
a buttress extending generally radially inwardly and axially along the outflow portion relative to the circumferential path, the buttress having a surface dimensioned and configured to be engaged by at least one leaflet of a heart valve when the automatic repair device is implanted at the heart valve, whereby when the automatic repair device is implanted at the heart valve, movement of the at least one leaflet of the heart valve relative to the surface of the buttress provides substantially unidirectional flow of blood relative to the apparatus,
wherein the fixation support member and buttress are deformable from the deployed configuration to any other configuration and automatically expandable again to the deployed configuration.

16. A method for implanting an automatic repair device, comprising:
compressing an automatic repair device to reduce an overall diameter of the automatic repair device;
loading the compressed automatic repair device into an implanter;
inserting the loaded implanter into a heart of a patient;
depositing the compressed automatic repair device into an annulus of a heart valve;
expanding the automatic repair device in the annulus such that the automatic repair device is implanted in the annulus; and
removing the implanter from the patient.

17. The method of claim 16, wherein the automatic repair device comprises:
a fixation support member having
an annular support frame formed from a plurality of support features, the plurality of support features including inflow support features and outflow support features, the inflow support features extending in an inflow direction from a circumferential path and the outflow support features extending in an outflow direction from the circumferential path, and
a support member web at least partially encasing the annular support frame to provide at least an inflow contact surface and an outflow contact surface,
wherein the outflow support features are shorter than the inflow support features;
a buttress having
a buttress support frame, and
a buttress web at least partially encasing the buttress to form a surface against which at least one leaflet of a heart valve may engage the buttress,
wherein the buttress is secured to the annular support frame along the circumferential path such that the buttress extends in the outflow direction;
wherein the fixation support member and buttress are flexible between a compressed configuration and an expanded configuration.

18. The method of claim 16, wherein the implanter includes a catheter or trocar.

19. The method of claim 16, wherein the loaded implanter is inserted through an apex of the heart.

20. The method of claim 16, wherein the automatic repair device automatically expands in the annulus after deposited in the annulus.

21. The method of claim 16, wherein the implantation is performed percutaneously.

22. An automatic repair device comprising:
a fixation support member having
an annular support frame formed from a plurality of support features, the plurality of support features including inflow support features defining an inflow portion and outflow support features defining an outflow portion, the inflow support features extending in an inflow direction from a circumferential path and the outflow support features extending in an outflow direction from the circumferential path, and
a support member web at least partially encasing the annular support frame to provide at least an inflow contact surface and an outflow contact surface,
wherein the outflow support features are shorter than the inflow support features, the outflow support features vary in length, and the inflow support features are uniform in length;
a buttress having
a buttress support frame, and
a buttress web at least partially encasing the buttress to form a surface against which at least one leaflet of a heart valve may engage the buttress,
wherein the buttress is secured to the annular support frame along the circumferential path such that the buttress extends in the outflow direction;
wherein the fixation support member and buttress are flexible between a compressed configuration and an expanded configuration.

23. The automatic repair device of claim 22, wherein, along the circumferential path, the outflow support features shorten in length and then lengthen such that a shortest outflow support feature is oppositely oriented from a longest support feature.

24. An automatic repair device comprising:
a fixation support member having an inflow portion and an outflow portion each extending radially outwardly and oppositely from a central annular portion to create an inflow umbrella and an outflow umbrella, wherein the inflow umbrella extends uniformly from the central annular portion, and wherein the outflow umbrella does not extend uniformly from the central annular portion and an overall length of the outflow umbrella is shorter than the inflow umbrella; and
a buttress, having a contact surface, secured along the central annular portion, the buttress extending away from the central annular portion and within the outflow umbrella,
wherein the fixation support member and buttress are flexible between a compressed configuration and an expanded configuration.
